(19) United States
(12) Patent Application Publication
(10) Pub. No.: US 2022/0356467 A1
(43) Pub. Date: Nov. 10, 2022

(54) METHODS FOR DUPLEX SEQUENCING OF CELL-FREE DNA AND APPLICATIONS THEREOF

(71) Applicant: Board of Regents, The University of Texas System, Austin, TX (US)

(72) Inventors: Rajyalakshmi LUTHRA, Houston, TX (US); Dzifa Y. DUOSE, Houston, TX (US); Scott KOPETZ, Houston, TX (US); Ignacio I. WISTUBA, Houston, TX (US); Stephanie ZALLES, Houston, TX (US); Saradhi MALLAMPATI, Houston, TX (US)

(73) Assignee: Board of Regents, The University of Texas System, Austin, TX (US)

(21) Appl. No.: 17/620,605

(22) PCT Filed: Jun. 25, 2020
(86) PCT No.: PCT/US2020/070181

§ 371 Date: Dec. 17, 2021

Related U.S. Application Data
(60) Provisional application No. 62/866,130, filed on Jun. 25, 2019.

(51) Int. Cl. C12N 15/10; C12Q 1/6806; C40B 40/06

(52) U.S. Cl. CPC C12N 15/1093; C12Q 1/6806; C40B 40/06

(57) ABSTRACT

Provided herein are methods of preparing cell-free DNA (cfDNA) for sequencing such that variant allele frequencies are maintained. Also provided are sequencing libraries prepared according to such methods. In addition, methods are provided for analyzing sequencing reads to determine variant allele frequencies. These methods may be used for diagnosing and/or evaluating cancer patients.

### REFERENCE TO RELATED APPLICATIONS

The present application claims the priority benefit of U.S. provisional application No. 62/866,130, filed Jun. 25, 2019, the entire contents of which is incorporated herein by reference.

### STATEMENT REGARDING FEDERALLY SPONSORED RESEARCH

This invention was made with government support under grant number CA184843 awarded by the National Institutes of Health. The government has certain rights in the invention.

### BACKGROUND

### 1. Field

The present invention relates generally to the fields of molecular biology and medicine. More particularly, it concerns methods of sequencing and analyzing selected genetic loci to identify variant allele frequencies.

### 2. Description of Related Art

Liquid biopsy-based molecular profiling has been shown to elucidate comprehensive genomic abnormalities present in both the primary tumor and distant metastases (Lebofsky et al., 2015; Pereira et al., 2017; Schrock et al., 2018). However, numerous technical challenges remain in the development of liquid biopsy-based molecular testing for clinical applications (Ma et al., 2015; Castro-Giner et al., 2018). Tumor cells undergoing apoptosis or necrosis or through active secretion tend to release DNA fragments into circulation, approximately 166 bp or less, and these fragments are often referred to as circulating tumor DNA (Wan et al., 2017; Stroun et al., 2001; Thierry et al., 2016; Underhill et al., 2016). In plasma, circulating tumor DNA is diluted into an abundant cell-free DNA (cfDNA) fraction arising from non-tumor cells. Capturing and retaining the much less abundant circulating tumor DNA fraction from total cfDNA throughout all the stages involved in the preparation of sequencing-ready libraries is challenging.

Background errors originate predominantly from DNA-damaging events to which the sample is subjected during extraction, library generation, or sequencing (Castro-Giner et al., 2018; Robasky et al., 2014; Williams et al., 1999; Park et al., 2017; Arbeithuber et al., 2016; Bruskov et al., 2002). These background errors can potentially contribute to false positive variants, and they tend to occur most frequently at low allelic frequencies (Kamps-Hughes et al., 2018; Newman et al., 2016). Because tumor-derived cfDNA constitutes only a minor fraction of the total cfDNA pool in the plasma, it is highly likely that mutations present in the tumor-derived cfDNA also occur at lower allelic frequencies (Lanman et al., 2015). Therefore, accurately distinguishing a true variant from a background error which also can be present at low frequency, poses another technical challenge in developing cfDNA-based molecular diagnostics for clinical applications (Salk et al., 2018).

Colorectal cancer is the third most frequently diagnosed cancer type worldwide and the second leading cause of cancer-related deaths. In approximately 21% of patients, this disease is diagnosed when it has already metastasized to the lungs, liver, and lymph nodes. Primary treatment options include chemotherapy, with less than 10% response rate (Foubert et al., 2014). In these patients, the disease is monitored essentially using conventional diagnostic imaging technologies, such as magnetic resonance imaging (MM) and computed tomography (CT) scan. To evaluate disease progression in patients with metastases, imaging analysis of distant organs is required. In contrast, a single cfDNA-based molecular test is theoretically able to provide a comprehensive assessment of disease status for the whole body. Therefore, liquid biopsy-based monitoring of disease in colorectal cancer patients potentially can offer an unprecedented advantage compared with traditional imaging-based approaches (Hao et al., 2014; Cassinotti et al., 2013; Kidess et al., 2015; Scholer et al., 2017; Tie et al., 2018; Tie et al., 2015; Christensen et al., 2018; Zhou et al., 2016).

### SUMMARY

In one embodiment, provided herein are methods of preparing a library of cell-free DNA (cfDNA) for sequencing, the method comprising: (a) obtaining a sample comprising a plurality of cfDNA; (b) performing end-repair and A-tailing reactions on between about 5 ng and about 30 ng of the plurality of cfDNA in a reaction having a first reaction volume; (c) contacting between about 2.5 ng and about 15 ng of the plurality of cfDNA with a population of stem-loop adaptors and a ligase in a second reaction volume that is about equal to the first reaction volume, wherein the stem-loop adaptors each comprise an inverted repeat and a loop, wherein the loop comprises at least one cleavable base, thereby ligating a stem-loop adaptor to each end of the plurality of cfDNA to produce adaptor-ligated cfDNA; (d) linearizing the adaptor-ligated cfDNA by cleaving the cleavable base; (e) amplifying the linearized adaptor-ligated cfDNA to produce amplified adaptor-ligated cfDNA, wherein the amplification uses forward and reverse primers complementary to known sequences in the stem-loop adaptors; (f) contacting the amplified adaptor-ligated cfDNA with RNA baits that hybridize to selected molecules of the plurality of cfDNA, wherein the weight ratio of RNA baits:amplified adaptor-ligated cfDNA is between about 1:25 and about 1:250; (g) isolating the molecules of the plurality of cfDNA having a hybridized RNA bait, thereby producing enriched cfDNA; and (h) amplifying the enriched cfDNA with indexing primers, thereby producing a library of cfDNA for sequencing.

In some aspects, the methods maintain variant allele frequencies in the cfDNA. In some aspects, the cfDNA comprises double-stranded DNA molecules. In some aspects, the cfDNA is obtained from a body fluid. In some aspects, the body fluid comprises blood, serum, urine, cerebrospinal fluid, nipple aspirate, sweat, or saliva. In some aspects, the cfDNA is obtained from an individual having a cancer.

In some aspects, end repair comprises exposing the plurality of cfDNA to a terminal deoxynucleotidyltransferase and an adenine deoxyribonucleotide. In some aspects, the stem-loop adaptors comprise a 3′ T overhang. In some aspects, the stem-loop adaptors comprise a 3′ hydroxyl and a 5′ phosphate.

In some aspects, the population of stem-loop adaptors comprises 75 ng of stem-loop adaptors. In some aspects, the stem-loop adaptors each comprise a constant region having a known sequence that is constant among the population of stem-loop adaptors and a barcode region having a sequence that is degenerate among the population of stem-loop adaptors. In some aspects, the barcode region is 4 nucleotides to 20 nucleotides in length. In some aspects, the barcode region is 13 or 14 nucleotides in length. In some aspects, the barcode regions is dephased. In some aspects, a portion of the population of stem-loop adaptors comprises a 13 nucleotide barcode region and another portion of the population of stem-loop adaptors comprises a 14 nucleotide barcode region. In some aspects, the portion comprising a 13 nucleotide barcode and the portion comprising a 14 nucleotide barcode are present at a 1:1 ratio. In some aspects, the barcode region is in the inverted repeat. In some aspects, the barcode regions are sufficiently unique so that each tagged double-stranded cfDNA molecule can be differentiated from other tagged double-stranded cfDNA molecules. In some aspects, the barcode regions of the stem-loop adaptors attached to each end of a cfDNA molecule comprise unique sequences.

In some aspects, the cleavable base is deoxyuridine. In some aspects, the cleavable base is cleaved prior to step (e). In some aspects, step (f) further comprises contacting the amplified adaptor-ligated cfDNA with adaptor blockers.

In some aspects, the RNA baits hybridize to selected genomic loci in a reference genome. In some aspects, the hybridization of the RNA baits to the cfDNA selectively enriches the cfDNA for strands that map to said genomic loci. In some aspects, the selected genomic loci comprise disease-associated genetic loci. In some aspects, the selected genomic loci comprise cancer-associated genetic loci. In some aspects, the selected genomic loci are in genes selected from the group consisting of TP53, APC, ATM, KRAS, NRAS, BRAF, PIK3CA, EGFR, NF1, NRAS, PDGFRA, PTEN, SMAD4, and ERBB2.

In some aspects, the RNA baits are oligonucleotides between about 70 nucleotides and 1000 nucleotides in length. In some aspects, the target-specific sequences in the RNA baits are between about 100 and about 200 nucleotides in length. In some aspects, the RNA baits have sequences that hybridize to a target sequence for at least 50, 75, 100, 125, 150, 175, 200, 225, or 250 of the genomic loci listed in Table 1. In some aspects, the RNA baits have sequences that hybridize to a target sequence for all 274 of the genomic loci listed in Table 1. In some aspects, the RNA baits have sequences that hybridize to a sequence in at least 10 of the genes listed in Table 1. In some aspects, the RNA baits have sequences that hybridize to a sequence in all 23 of the genes listed in Table 1.

In some aspects, the RNA baits each comprise an affinity tag. In some aspects, the affinity tag is a biotin molecule or a hapten.

In some aspects, step (g) comprises contacting the hybridized molecules from step (f) with a molecule or particle that binds to the RNA baits and isolating the RNA bait sequences, thereby isolating the subgroup of cfDNA molecules that hybridized to the RNA baits. In some aspects, the molecule or particle that binds to the RNA baits binds to the affinity tag. In some aspects, the molecule or particle that binds to the RNA baits is an avidin molecule or an antibody that binds to the hapten.

In some aspects, amplifying in step (e) and/or (h) comprises performing polymerase chain reaction.

In one embodiment, provided herein are libraries of cfDNA molecules generated by the method of any one of the present embodiments.

In one embodiment, provided herein are methods of analyzing the library of cfDNA molecules, comprising (a) sequencing the library of cfDNA. In some aspects, the methods further comprise (b) generating a single consensus sequence for each forward and reverse sequence by grouping all sequencing reads that share the same variant adaptor sequences on both their 5′ and 3′ ends, representing each position in the consensus sequence with the nucleotide present in the sequencing reads only if all sequencing reads in the family have the same nucleotide at that position, representing each position in the consensus sequence with N if the sequencing reads in the family have different nucleotides at that position.

In some aspects, the methods further comprise generating a double consensus sequence by (a) identifying a reverse single consensus sequence having a molecular barcode in reverse orientation relative to a molecular barcode for a given forward single consensus sequence, representing each position in the double consensus sequence with the nucleotide present in both the forward SCS and reverse SCS reads only if the forward SCS and reverse SCS have the same nucleotide at that position, representing each position in the DCS with N if the forward SCS and the reverse SCS have different nucleotides at that position; and (b) identifying a forward single consensus sequence having a molecular barcode in reverse orientation relative to a molecular barcode for a given reverse single consensus sequence, representing each position in the double consensus sequence with the nucleotide present in both the forward SCS and reverse SCS reads only if the forward SCS and reverse SCS have the same nucleotide at that position, representing each position in the DCS with N if the forward SCS and the reverse SCS have different nucleotides at that position.

In some aspects, the methods further comprise aligning the single consensus sequences derived from families containing at least two reads with a human reference genome and identifying variants in the single consensus sequences. In some aspects, the methods further comprise aligning the double consensus sequences with a human reference genome and identifying variants in the double consensus sequences.

In some aspects, the methods further comprise detecting a copy number variation in the cfDNA, wherein the copy number variation is based at least on part on the quantification of the sequencing reads that map to each of one or more genetic loci. In some aspects, the methods further comprise quantifying cfDNA molecules bearing a sequence variant.

In some aspects, quantifying cfDNA molecules bearing a sequence variant comprises only counting the variant allele if the variant allele count was at least 4. In some aspects, quantifying cfDNA molecules bearing a sequence variant comprises only counting the variant allele if the read balance ratio was at least 0.1. In some aspects, quantifying cfDNA molecules bearing a sequence variant comprises only counting the variant allele if the ratio of variant frequency in the sample is more than two-fold different than a variant frequency in a healthy control sample.

In one embodiment, provided herein are methods of monitoring progression of cancer in a patient, monitoring response to therapy in a cancer patient, or detecting minimum residual disease in a cancer patient, the method comprising analyzing cfDNA obtaining from the patient at at least two time points according to the method of any one of the present embodiments and comparing the variant allele frequencies at the at least two time points. In some aspects, the patient has colorectal cancer, ovarian cancer, lung cancer, prostate cancer, liver cancer, kidney cancer, pancreatic cancer, uterine cancer, brain cancer, skin cancer, stomach cancer, or breast cancer.

In one embodiment, provided herein are compositions comprising a set of RNA baits that hybridize to a target sequence for at least 50 of the genomic loci listed in Table 1. In some aspects, the composition comprises RNA baits that hybridize to the target sequence for at least 100, 150, 200, or 250 of the genomic loci listed in Table 1. In some aspects, the composition comprises RNA baits that hybridize to the target sequence of all 274 of the genomic loci listed in Table 1. In some aspects, the composition comprises RNA baits that hybridize to a sequence in at least 10 of the genes listed in Table 1. In some aspects, the composition comprises RNA baits that hybridize to a sequence in all 23 of the genes listed in Table 1. In some aspects, the RNA baits each comprise an affinity tag.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The key library preparation stages of the CRC23 assay. The following steps were performed sequentially: cfDNA end repair and dA-tailing, hairpin adaptor ligation, uracil base excision, first PCR amplification, hybridization capture of target regions, and second PCR amplification. The second PCR amplification was performed using primers containing sample indexes, and those indexed samples were pooled and sequenced on Nextseq550.

FIGS. 2A-B. Optimization of enrichment baits improves hybridization capture efficiency. (A, B) Differential read counts indicate relative depth in the coverage of target regions at any two tested concentrations (FIG. 2A shows 500 ng vs. 180 ng, 180 ng vs. 60 ng, and 60 ng vs. 30 ng; FIG. 2B shows 60 ng vs. 30 ng, 30 ng vs. 15 ng, and 15 ng vs 7.5 ng) of enrichment baits. Note that the absolute read counts from individual target regions were normalized and denoted as read counts per 100-bp target region. Differential read counts were calculated by subtracting normalized read counts from two successive bait concentrations used in the assay. In each individual comparison, normalized read counts from high concentration of baits were regarded as control and those from low concentration as test entities.

FIGS. 3A-B. Optimization of critical steps involved in the library preparation improves accuracy of identified variant allele frequencies. Evaluation of first (FIG. 3A) and second (FIG. 3B) PCR amplification products with BRAF V600E droplet digital PCR assay. Note that the observed BRAF V600E frequency in the original cfDNA was similar to the frequencies observed in the amplification products from the fourth library preparation workflow.

FIGS. 4A-F. Structure of adaptor facilitates acquisition of single or dual molecular barcodes into the sequencing library templates. The structure of single (FIG. 4A) or dual (FIG. 4C) molecular barcode yielding adaptors. Evaluation of sequencing libraries prepared from single (FIG. 4B) or dual (FIG. 4D) molecular barcode adaptors with the sequence analysis viewer. (FIG. 4E) Relative fraction of consensus reads that contain stretches of ‘N’. Note that each consensus read was generated from the group of reads sharing the same molecular barcode index and the presence of 8 or 10 consecutive ‘N’s in that derived consensus read was denoted as stretch. (FIG. 4F) Illumina sequencing analysis viewer visualization of libraries that were prepared utilizing a unique adaptor mix and sequenced on Nextseq. Note that in any given sequencing cycle of depicted run A, C, G or T individual nucleotides frequency is lower than 65% indicating that the adaptor mix was able to confer base diversity to library without addition of PhiX control.

FIGS. 5A-C. Evaluation of CRC23 assay analytical performance. (FIG. 5A) Sequencing coverage depth across different variant positions of the mutant cfDNA pool diluted at 10% frequency. (FIG. 5B) Distribution of mutant allele frequencies identified from the mutant cfDNA pool diluted at 1% frequency. (FIG. 5C) Determination of the limit of detection (LOD) of the CRC23 assay. Note that the variants that were expected to occur between 0.3-0.39%, 0.2-0.29%, and 0.1-0.19% frequencies were evaluated for determining LOD.

FIGS. 6A-C. Evaluation of clinical diagnostic performance of the CRC23 assay. (FIG. 6A) Identification of most frequently mutated genes in colorectal cancer patients. Note that within the 27 colorectal cancer cfDNA samples sequenced, TP53, KRAS, and APC genes are frequently mutated. (FIG. 6B) Correlation of the frequencies of common mutant alleles identified from the CRC23 and Guardant360 assays. Observed MAF findings were from CRC23 assay and the expected MAF findings were from the Guardant360 assay. (FIG. 6C) Mutant allele frequencies derived from single consensus sequences (SCS) and double consensus sequences (DCS) indicate a high degree of concordance.

FIGS. 7A-F. Monitoring of disease progression in stage IV colorectal cancer patients with the CRC23 assay. (FIGS. 7A, 7B, 7C, 7D, 7F) Trends in mutant allele frequencies correlate with the disease status observations obtained from imaging. Note that from each patient, three plasma samples were collected at different time points. Imaging was performed at multiple points, and these patients were subjected to multiple treatment regimens. Inner tick at the bottom of the image indicates the point of blood sample collection; the vertical line indicates the point of imaging; the shaded area represents the period for which the patient was subjected to a treatment regimen; the arrow at the top of the image provides clinical interpretations obtained from imaging. (FIG. 7E) Confirmation of the newly evolved variants through duplex sequencing.

FIG. 8. Schematic outline of the strategy for double consensus sequence (DCS) derivation from duplex sequencing. During library preparation, P5 adaptor and molecular barcode sequences (referred to here as ‘α’ and ‘β’) are ligated to the 5′ ends of the top and bottom strands of input cfDNA. The 3′ ends of the top and bottom strands receive the complementary sequences of the ‘β’ and ‘α’ molecular barcodes, respectively, along with P7 adaptor sequences. During PCR amplification, each strand produces its complementary sequence; the top strand (depicted in blue) yields a complementary sequence depicted in black, and the bottom strand (depicted in red) produces a complementary sequence depicted in yellow. After paired-end sequencing, the first 14 bp molecular barcode information from a sequencing read in the forward-reads file (denoted here with ‘F’) and corresponding sequencing read in the reverse-reads file (denoted here with ‘R’) are combined computationally and used as an index for these sequencing reads (denoted here as ‘αβ’ or ‘βα’). Sequencing reads that share the same molecular barcode index are grouped together, and from each group of reads a single consensus sequence (SCS) is derived. To derive a DCS, the SCS read containing the ‘αβ’ molecular barcode index from the forward-read file is grouped with the SCS read containing the ‘βα’ molecular barcode index from the reverse-read file. In a similar manner, the SCS read containing the ‘αβ’ molecular barcode index from the reverse-read file is grouped with the SCS read containing the ‘βα’ molecular barcode index from the forward-read file.

FIG. 9. A position-specific error model that effectively aids in correcting sequencing errors accrued in patient cfDNA. A position-specific variant allele frequency (error) model was created by sequencing cfDNA isolated from the plasma samples of healthy controls. Gaussian distribution of variant allele frequencies observed in these control cfDNA samples was used to evaluate the specificity of variant frequencies noticed in the patient cfDNA samples. If the variant frequencies in the patient cfDNA samples were within the limits of the Gaussian distribution of variant frequencies from the control cfDNA, the variant allele frequencies in the patients' cfDNA were considered an error and adjusted to zero. In the example shown, the reference base ‘A’ (indicated in a box) was mutated to G′ and ‘T’ in the controls. In the patient sample (Test), the same reference base was mutated to ‘G.’ Evaluation of this mutant allele frequency (MAF) on the basis of the Gaussian distribution of MAFs in the controls identified it as an error; therefore, the frequency was adjusted to zero.

FIGS. 10A-E. Distribution of variant allele frequencies in the mutant cfDNA pool diluted at 10% (FIG. 10A), 2% (FIG. 10B), 1.5% (FIG. 10C), 0.5% (FIG. 10D), and 0.2% (FIG. 10E) proportions. For each mutant cfDNA dilution, triplicate samples were sequenced. Each outward mark on the x-axis denotes a variant. For each variant, individual frequencies and their mean frequency values are shown.

### DETAILED DESCRIPTION

Potential applications of cell-free DNA (cfDNA)-based molecular profiling have been shown in diverse malignancies. However, capturing all cfDNA originating from tumor cells and identifying true variants present in this minute fraction of cfDNA remain a key challenge to widespread applications of cfDNA-based liquid biopsies in the clinical setting. Provided here is a systematic approach and key components of wet bench and bioinformatics strategies to address these challenges. The concentration of enrichment oligonucleotides, elements of the library preparation, and the structure of adaptors are critical for achieving high enrichment of the target regions, retaining the variant allele frequencies accurately throughout all involved steps of library preparation, and obtaining high variant coverage. A dual molecular barcode integrated error elimination strategy removes sequencing artifacts, an optimized alignment approach identifies low frequency variants, and a background error correction strategy distinguishes true variants from abundant false-positive variants. Further, a clinical application of this cfDNA-based duplex sequencing approach is provided through monitoring disease progression in patients with stage IV colorectal cancer. These cfDNA-based molecular testing observations are highly concordant with observations obtained by traditional imaging methods. The methods provided herein can be used for the early detection of cancer, identifying minimal residual disease, and the evaluation of therapeutic responses in cancer patients. For example, this cfDNA-based molecular assay can be used to monitor disease progression in patients with stage IV colorectal cancer using the provided colorectal cancer-specific next-generation sequencing (NGS) panel.

### I. ASPECTS OF THE PRESENT EMBODIMENTS

Provided herein is a systematic approach for developing a cfDNA-based molecular test for liquid biopsies. Provided are critical steps involved in both the wet bench methods and the bioinformatics pipeline.

Theoretically, a hybridization capture-based approach compared with an amplicon-based approach is a better choice for cfDNA-based liquid biopsy applications (Lanman et al., 2015; Samorodnitsky et al., 2015; Garcia-Garcia et al., 2016). Tumor cells release cfDNA fragments into the circulation through apoptosis, necrosis, or active secretion (Wan et al., 2017; Stroun et al., 2001; Thierry et al., 2016). Irrespective of their mode of release, these fragments seem to be generated from a random fragmentation process. Each fragment contains a distinct beginning and end. In an amplicon-based method, if the variants of interest are present at the edges of the randomly generated cfDNA templates, these fragments might not yield any amplification because they lack a binding sequence for any of the primers. In contrast, a hybridization capture-based approach could enrich for these types of cfDNA fragments effectively, as the binding of probe to targeted region or adjoining region would be sufficient to capture the variant. In the hybridization capture approach, the capture size varies from a few kilobases to several megabases (Samorodnitsky et al., 2015). Increase in the size of capture is positively correlated with on-target enrichment percentages. In this study, a panel that covers 78.81-Kb target regions was designed. With this size panel, the obtainable on-target percentages are projected to be less than 50%. To improve the on-target enrichment percentage without compromising absolute coverage of individual target regions, enrichment bait concentrations during hybridization capture were optimized and significant improvement was seen when baits concentrations were below 10 ng. Depending on the capture size, optimization of enrichment bait concentration can yield significantly better on-target recovery.

Most of the commercially available NGS library preparation methods have been tailored for tissue biopsy specimens and aim to identify variants occurring at frequencies of 5% and above. However, in cfDNA-based liquid biopsy applications, the ability to identify variants that occur below 1% frequency is critical (Newman et al., 2016; Lanman et al., 2015). A good library preparation protocol must maintain variant allele frequencies of the original cfDNA pool throughout all the stages of library preparation. In this study, a library preparation strategy that accurately facilitates identification of ultralow frequency variants was developed.

In this study, two versions of adaptors were evaluated and unique advantages of using dual molecular barcode adaptors over single molecular barcode adaptors for cfDNA-based applications were shown. In the case of dual barcode adaptors, two 14-bp molecular barcode sequences are integrated and a single 28-bp molecular barcode that is derived with a bioinformatics strategy. Therefore, the unique molecular barcode diversity that could be obtained with dual barcode adaptors was thousands-fold higher than with single barcode adaptors. For this reason, the fraction of diverse templates receiving the same molecular barcode remains higher in the case of a single molecular adaptor, and a higher fraction of unusable consensus sequence reads was observed when a single molecular adaptor was utilized. More importantly, dual molecular barcode adaptors facilitated duplex sequencing of cfDNA templates (Schmitt et al., 2012). Sequencing artifacts can arise randomly or in a recurrent manner and contribute to low allelic frequency variants, which are often regarded as false positives (Kamps-Hughes et al., 2018; Newman et al., 2016). However, a random variant is unlikely to occur at the same position on both top and bottom strands of cfDNA. Therefore, if a variant is observed in both template strands it is more likely to be a true variant (Schmitt et al., 2012). For this reason, duplex sequencing was used to identify a variant that was present in both top and bottom strands of cfDNA. In duplex sequencing, consensus reads are derived in two stages. In the first stage, SCS reads are derived from the original sequencing reads, and in the second stage DCS reads are derived using SCS reads as a template. In this study, for variant identification purposes, SCS reads were used. Variants identified from DCS reads are used only under circumstances when further verification of the identified variant from SCS reads is required. Further advancement in the current technology will allow using DCS reads in place of SCS reads for variant identification.

A 78.81-kb colorectal cancer—specific panel was designed based on variant information retrieved from approximately 3,000 patient samples. Using this panel, 85% of variants present in this cohort could be identified. In the 27 CRC samples sequenced, TP53, APC, and KRAS were identified as the most frequently mutated, and indeed, these genes have been shown to be the key players in this cancer type (Strickler et al., 2018). These sequencing findings were compared with the findings obtained after sequencing of these samples with the Guardant360 assay as an orthogonal method (Lanman et al., 2015). Frequencies of variant alleles that were detected in both assays showed high concordance. However, six variants that were exclusively identified in the Guardant360 assay were identified. This discrepancy is potentially explained by pre-analytical variables that differ between the two assays (Mehrotra et al., 2017). In the Guardant360 assay, blood samples were collected in Streck tubes and extractions were performed with an automated version of the protocol that utilizes magnetic bead-based extraction. In this assay, blood samples were drawn in EDTA Vacutainer tubes and cfDNA was extracted using column-based manual extraction protocols. As a result, significant amounts of high-molecular-weight genomic DNA contamination were observed in the manually extracted cfDNA (Norton et al., 2013), and an additional size selection step was incorporated following extraction for excluding genomic DNA. Although high-quality cfDNA were obtained after size selection the total amount of cfDNA that was used for library preparation might be lower than the quantities used in the Guardant360 assay as a consequence of losses incurred during the size selection process. Because the lower limit of detection of an assay is directly proportional to input cfDNA, the lower inputs of cfDNA utilized in this assay could possibly explain exclusive variants identified in the Guardant360 assay.

The present assay was clinically applied by monitoring disease progression in patients with stage IV colorectal cancer. The cfDNA sequencing of the longitudinal samples collected from these patients showed that mutant allele frequency trends in the samples were concordant with imaging observations. When the trend of mutant allele frequencies was compared between the current and previous collection specimens, the increases in the mutant allele frequencies in the current collection were correlated with disease progression. On the other hand, decreased mutation frequencies were observed that correlated with regressed tumor foci at metastatic sites or stable disease. Tumor-released cfDNAs have a half-life of 16 minutes to 2.5 hours (Wan et al., 2017; Diehl et al., 2008; To et al., 2003; Yao et al., 2016). Owing to its short half-life, cfDNA could be used for real-time tracing of tumor progression. However, caution needs to be exercised if monitoring samples are collected while the patient is under a treatment regimen, as tumor cell death releases cfDNA into circulation, and that would in turn also lead to an increase in the mutant allele frequency. Indeed, cfDNA-based molecular profiling has been shown to be sensitive in contrast to imaging-based approaches and was used in previous studies for monitoring disease progression in patients with melanoma and cancers of the breast, lung, pancreas, and colon (Takai et al., 2015; Guo et al., 2016; Hench et al., 2018; Shu et al., 2017; Bettegowda et al., 2014; Abbosh et al., 2017). New variants that were identified exclusively in later time points and not in earlier time points, and the variants that were present in earlier collections and absent in subsequent collections, were verified through duplex sequencing strategy. Therefore, in cfDNA-based molecular profiling applications, duplex sequencing undoubtedly increases the accurate identification of variants that might emerge or diminish during the course of longitudinal monitoring. Furthermore, the variants that were observed at low frequencies were often increased significantly in collections made at later time points, emphasizing the point that identification of low-frequency variants is critical for cfDNA-based molecular testing and that their early identification can have a potential effect on disease management (Wan et al., 2017).

In conclusion, the approaches presented here have potential utility towards applications involving cfDNA-based molecular profiling for early detection of cancer, identification of minimal residual disease, and the evaluation of therapeutic responses in cancer patients (Frenel et al., 2015; Thierry et al., 2017; Anker & Stroun, 2001; Tie et al., 2016; Heitzer et al., 2017).

### II. DEFINITIONS

The term “subject” or “patient” as used herein refers to any individual to which the subject methods are performed. Generally the patient is human, although as will be appreciated by those in the art, the patient may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The methods described herein are useful in treating cancer. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

A response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

The terms “hairpin,” “stem-loop adaptor,” and “stem-loop oligonucleotide,” as used herein, refer to a structure formed by an oligonucleotide comprised of 5′ and 3′ terminal regions, which are inverted repeats that form an at least partially double-stranded stem, and a non-self-complementary central region, which forms a single-stranded loop. In some embodiments, the stem-loop oligonucleotide further comprises a second or third single-stranded loop, such as within the 5′ stem and/or the 3′ stem. An “asymmetric loop” refers to a single-stranded loop on only one stem strand with a “gap region” of unpaired bases across from the asymmetric loop.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.” In addition, the term “adaptor” can indicate a linear adaptor (either single stranded or double stranded) or a stem-loop adaptor. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

“Cleavable base,” as used herein, refers to a nucleotide that is generally not found in a sequence of DNA. For most DNA samples, deoxyuridine is an example of a cleavable base. Although the triphosphate form of deoxyuridine, dUTP, is present in living organisms as a metabolic intermediate, it is rarely incorporated into DNA. When dUTP is incorporated into DNA, the resulting deoxyuridine is promptly removed in vivo by normal processes, e.g., processes involving the enzyme uracil-DNA glycosylase (UDG) (U.S. Pat. No. 4,873,192; Duncan, 1981; both references incorporated herein by reference in their entirety). Thus, deoxyuridine occurs rarely or never in natural DNA. Non-limiting examples of other cleavable bases include deoxyinosine, bromodeoxyuridine, 7-methylguanine, 5,6-dihyro-5,6 dihydroxydeoxythymidine, 3-methyldeoxadenosine, etc. (see, Duncan, 1981). Other cleavable bases will be evident to those skilled in the art.

The term “degenerate” as used herein refers to a nucleotide or series of nucleotides wherein the identity can be selected from a variety of choices of nucleotides, as opposed to a defined sequence. In specific embodiments, there can be a choice from two or more different nucleotides. In further specific embodiments, the selection of a nucleotide at one particular position comprises selection from only purines, only pyrimidines, or from non-pairing purines and pyrimidines.

The term “ligase” as used herein refers to an enzyme that is capable of joining the 3′ hydroxyl terminus of one nucleic acid molecule to a 5′ phosphate terminus of a second nucleic acid molecule to form a single molecule. The ligase may be a DNA ligase or RNA ligase. Examples of DNA ligases include E. coli DNA ligase, T4 DNA ligase, and mammalian DNA ligases.

The term “molecular barcode” as used herein refers to a unique nucleotide sequence that is used to distinguish duplicate sequences arising from amplification from those which are molecular barcode can be linked to a target nucleic acid of interest by ligation prior to amplification, or during amplification (e.g., reverse transcription or PCR), and used to trace back the amplicon to the genome or cell from which the target nucleic acid originated. A molecular barcode can be added to a target nucleic acid by including the sequence in the adaptor to be ligated to the target. A molecular barcode can also be added to a target nucleic acid of interest during amplification by carrying out reverse transcription with a primer that contains a region comprising the barcode sequence and a region that is complementary to the target nucleic acid such that the barcode sequence is incorporated into the final amplified target nucleic acid product (i.e., amplicon). The molecular barcode may be any number of nucleotides of sufficient length to distinguish the molecular barcode from other molecular barcodes. For example, a molecular barcode may be anywhere from 4 to 20 nucleotides long, such as 5 to 11, or 12 to 20.

“Sample” means a material obtained or isolated from a fresh or preserved biological sample or synthetically-created source that contains nucleic acids of interest. In certain embodiments, a sample is the biological material that contains the variable region(s) for which data or information are sought. Samples can include specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, or amniotic fluid. Samples can also include non-human sources, such as non-human primates, rodents and other mammals, other animals, plants, fungi, bacteria, and viruses.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

### III. MOLECULAR BARCODED STEM-LOOP ADAPTORS

The molecular barcode may be a double-stranded, complementary sequence. In some embodiments, the stem-loop adaptor molecule includes a molecular barcode sequence of nucleotides that is degenerate or semi-degenerate. In some embodiments, the degenerate or semi-degenerate molecular barcode sequence may be a random degenerate sequence. A double-stranded molecular barcode sequence includes a first degenerate or semi-degenerate nucleotide n-mer sequence and a second n-mer sequence that is complementary to the first degenerate or semi-degenerate nucleotide n-mer sequence. The first and/or second degenerate or semi-degenerate nucleotide n-mer sequences may be any suitable length to produce a sufficiently large number of unique tags to label a set of cfDNA fragments in a sample. Each n-mer sequence may be between approximately 3 to 20 nucleotides in length. Therefore, each n-mer sequence may be approximately 4, 5, 6, 7, 8, 9, 10, 11, 12, 13, 14, 15, 16, 17, 18, 19, 20 nucleotides in length. In one embodiment, the molecular sequence is a random degenerate nucleotide n-mer sequence which is 14 nucleotides in length. A 14 nucleotide molecular barcode n-mer sequence that is ligated to each end of a cfDNA molecule results in generation of up to 4²⁸ (i.e., 7.2×10¹⁶) distinct tag sequences.

The molecular barcode nucleotide sequence may be completely random and degenerate, wherein each sequence position may be any nucleotide. (i.e., each position, represented by “N,” is not limited, and may be an adenine (A), cytosine (C), guanine (G), thymine (T), or uracil (U)) or any other natural or non-natural DNA or RNA nucleotide or nucleotide-like substance or analog with base-pairing properties (e.g., xanthosine, inosine, hypoxanthine, xanthine, 7-methylguanine, 7-methylguanosine, 5,6-dihydrouracil, 5-methylcytosine, dihydouridine, isocytosine, isoguanine, deoxynucleosides, nucleosides, peptide nucleic acids, locked nucleic acids, glycol nucleic acids and threose nucleic acids). The term “nucleotide” as described herein, refers to any and all nucleotide or any suitable natural or non-natural DNA or RNA nucleotide or nucleotide-like substance or analog with base pairing properties as described above. In other embodiments, the sequences need not contain all possible bases at each position.

The stem-loop adaptor molecules are ligated to both ends of a target nucleic acid molecule, and then this complex is used according to the methods described below. The stem-loop adaptor may be any suitable ligation adaptor that is complementary to a ligation adaptor added to a double-stranded target nucleic acid sequence including, but not limited to a T-overhang, an A-overhang, a CG overhang, a blunt end, or any other ligatable sequence. In some embodiments, the stem-loop adaptor may be made using a method for A-tailing or T-tailing with polymerase extension; creating an overhang with a different enzyme; using a restriction enzyme to create a single or multiple nucleotide overhang, or any other method known in the art.

According to the embodiments described herein, the stem-loop adaptor molecule may include at least two PCR primer binding sites: a forward PCR primer binding site; and a reverse PCR primer binding site. The stem-loop adaptor molecule may also include at least two sequencing primer binding sites, each corresponding to a sequencing read. Alternatively, the sequencing primer binding sites may be added in a separate step by inclusion of the necessary sequences as tails to the PCR primers, or by ligation of the needed sequences. Therefore, if a double-stranded target nucleic acid molecule has a stem-loop adaptor molecule ligated to each end, each sequenced strand will have two reads—a forward and a reverse read.

Molecular barcode containing adaptor ligated DNA templates acquire C, T, T nucleotides at 5th, 10th, and 15th positions, respectively. As every template at these positions contains exactly the same base, the diversity of library at those positions is limited. In order to impart library diversity, a control library prepared from PhiX DNA was mixed with test samples DNA library up to 20% prior to sequencing. Sequencing performed using Nextseq high output flow cell typically yields up to 800 million reads; it means that sequencing of PhiX control library could consume approximately 160 million reads. In order to utilize most effectively the entire space on flow cell for sequencing test sample libraries, an adaptor cocktail that would preclude the need for adding control library prepared from PhiX DNA was designed. An additional adaptor, which contains 13 nucleotide molecular barcode (NNNCNNNNTNNNN), was prepared and mixed with adaptor containing 14 nucleotide molecular barcode CNNNNTNNNN) in 1:1 ratio to obtain ligation ready adaptor cocktail. The adaptor cocktail aided in reducing the C, T, T nucleotide base composition during 5th, 10th, and 15th cycles of sequencing from 100% to 62.5%; thus facilitated achieving the base diversity without supplementation of PhiX control library to the test sample libraries.

### IV. RNA BAIT HYBRIDIZATION

The selection methods of the invention may be carried out by hybridization in solution, i.e., neither the oligonucleotide bait sequences nor the group of nucleic acids (containing target nucleic acid molecules that are desired to be selected from the group of nucleic acids) being selected from are attached to a solid surface. Performing the selection method by hybridization in solution minimizes the reaction volume and therefore the amount of target nucleic acid necessary to achieve the concentration necessary to drive the hybridization reaction.

Prior to hybridization, baits can be denatured according to methods well known in the art. In general, hybridization steps comprise adding an excess of blocking DNA to the labeled bait composition, contacting the blocked bait composition under hybridizing conditions with the target sequences to be detected, washing away unhybridized baits, and detecting the binding of the bait composition to the target. The blocking DNA hybridizes to the known or substantially known stem-loop adaptor sequences.

Bait sequences preferably are oligonucleotides between about 70 nucleotides and 1000 nucleotides in length, more preferably between about 100 nucleotides and 300 nucleotides in length, more preferably between about 130 nucleotides and 230 nucleotides in length and more preferably still are between about 150 nucleotides and 200 nucleotides in length. Intermediate lengths in addition to those mentioned above also can be used in the methods of the invention, such as oligonucleotides of about 70, 80, 90, 100, 110, 120, 130, 150, 160, 180, 190, 210, 220, 230, 240, 250, 300, 400, 500, 600, 700, 800, and 900 nucleotides in length, as well as oligonucleotides of lengths between the above-mentioned lengths. For selection of exons and other short targets, preferred bait sequence lengths are oligonucleotides of about 100 to about 300 nucleotides, more preferably about 130 to about 230 nucleotides, and still more preferably about 150 to about 200 nucleotides. The target-specific sequences in the oligonucleotides for selection of exons and other short targets are between about 40 and 1000 nucleotides in length, more preferably between about 70 and 300 nucleotides, more preferably between about 100 and 200 nucleotides, and more preferably still between about 120 and 170 nucleotides in length. For selection of targets that are long compared to the length of the capture baits, such as genomic regions, preferred bait sequence lengths are typically in the same size range as the baits for short targets mentioned above, except that there is no need to limit the maximum size of bait sequences for the sole purpose of minimizing targeting of adjacent sequences.

In certain embodiments, bait sequences contain all sequences in the regions or targets of interest. In preferred embodiments, the bait sequences exclude certain sequences that are non-unique or repetitive in the genome. In preferred embodiments of hybrid selection in mammalian genomes such as the human genome, each bait contains less than 40 bases that are flagged as repetitive and/or low-complexity by algorithms and computer programs well known to those skilled in the art. In one preferred embodiment, the bait sequences are laid onto the reference sequence followed by removal of certain baits that contain less than the pre-defined limit of bases that are flagged as repetitive or low-complexity in whole-genome annotations. The baits can be laid onto the reference genome sequence such that neighboring baits overlap, such that there are no gaps or overlaps between adjacent baits, or such that there are gaps.

In some embodiments, the bait sequences in the set of bait sequences are RNA molecules. These can be made as described elsewhere herein, using methods known in the art, including de novo chemical synthesis and transcription of DNA molecules using a DNA-dependent RNA polymerase. The RNA molecules can be RNase-resistant RNA molecules, which can be made, for example, by using modified nucleotides during transcription to produce RNA molecules that resist RNase degradation. In preferred embodiments, RNA bait sequences include an affinity tag. In some embodiments, RNA bait sequences are made by in vitro transcription, for example, using biotinylated UTP. In other embodiments, RNA bait sequences are produced without biotin and then biotin is crosslinked to the RNA molecules using methods well known in the art, such as psoralen crosslinking.

As used herein, “group of nucleic acids” means nucleic acids that contain target sequences and are hybridized to bait sequences to select the target sequences. As used herein, “target sequences” are the set of sequences that one desires to isolate from the group of nucleic acids. The term target describes the scope or purpose of the experiment. To use the embodiment of exons as an example, the target sequences can be a specific group of exons, e.g., 500 particular exons. The target sequences, in a different example, can be all ^{˜}300,000 protein-coding exons in the human genome. The sequences that are actually selected from the group of nucleic acids is referred to herein as a “subgroup of nucleic acids”. The term subgroup describes the performance of the method, i.e., that not all of the target sequences are recovered by any particular use of the processes described herein. For example, the subgroup may in some embodiments be a percentage of the target sequences that is as low as 10% or as high as 90%.

The target sequences (and the subgroup of nucleic acids) obtained from genomic DNA can include a small fraction of the total genomic DNA, such that it includes less than about 0.0001%, at least about 0.0001%, at least about 0.001%, at least about 0.01% or 0.1% of genomic DNA, or a more significant fraction of the total genomic DNA, such that it includes at least: about 2% of genomic DNA, about 3% of genomic DNA, about 4% of genomic DNA, about 5% of genomic DNA, about 6% of genomic DNA, about 7% of genomic DNA, about 8% of genomic DNA, about 9% of genomic DNA, about 10% of genomic DNA, or more than 10% of genomic DNA.

In some embodiments, the bait set includes oligonucleotides that contain degenerate or mixed bases at one or more positions. In still other embodiments, the bait set includes multiple or substantially all known sequence variants present in a population of a single species or community of organisms. In one embodiment, the bait set includes multiple or substantially all known sequence variants present in a human population.

A large number of bait sequences may be used effectively in solution hybridization. A complex mixture of several thousand bait sequences can effectively hybridize to complementary nucleic acids in a group of nucleic acids and that such hybridized nucleic acids (the subgroup of nucleic acids) can be effectively separated and recovered. Thus it is possible in some embodiments to use a set of bait sequences containing more than 5,000 bait sequences, more than 6,000 bait sequences, more than 7,000 bait sequences, more than 8,000 bait sequences, more than 9,000 bait sequences, more than 10,000 bait sequences, more than 11,000 bait sequences, more than 12,000 bait sequences, more than 13,000 bait sequences, more than 14,000 bait sequences, more than 15,000 bait sequences, more than 16,000 bait sequences, more than 17,000 bait sequences, more than 18,000 bait sequences, more than 19,000 bait sequences, more than 20,000 bait sequences, more than 30,000 bait sequences more than 40,000 bait sequences more than 50,000 bait sequences more than 60,000 bait sequences more than 70,000 bait sequences more than 80,000 bait sequences more than 90,000 bait sequences, more than 100,000 bait sequences, or more than 500,000 bait sequences.

In embodiments, the method comprises sequencing, e.g., by a next generation sequencing method, a subgroup of nucleic acids from at least five, six, seven, eight, nine, ten, fifteen, twenty, twenty-five, thirty or more genes or gene products from the acquired cfDNA sample, wherein the genes or gene products are chosen from: ABL1, AKT1, AKT2, AKT3, ALK, APC, AR, BRAF, CCND1, CDK4, CDKN2A, CEBPA, CTNNB1, EGFR, ERBB2, ESR1, FGFR1, FGFR2, FGFR3, FLT3, HRAS, JAK2, KIT, KRAS, MAP2K1, MAP2K2, MET, MLL, MYC, NF1, NOTCH1, NPM1, NRAS, NTRK3, PDGFRA, PIK3CA, PIK3CG, PIK3R1, PTCH1, PTCH2, PTEN, RB1, RET, SMO, STK11, SUFU, or TP53, thereby analyzing the cfDNA.

In one embodiment, a panel of bait sequences may hybridize to the target sequences listed in Table 1. Such a panel may be used in methods of diagnosing and evaluating a colorectal cancer patient.

TABLE 1
CRC23 assay RNA bait target sequences. All position
numbers are relative to the GRCh37/hg19 genome version.

| | | | | NCBI RefSeq |
|---|---|---|---|---|
| | | | | Transcript |
| Gene | Chromo- | Start | End | Accession |
| Name | some | Position | Position | Number |
| NRAS | 1 | 115251155 | 115251275 | NM_002524.4 |
| NRAS | 1 | 115252189 | 115252349 | NM_002524.4 |
| NRAS | 1 | 115256420 | 115256599 | NM_002524.4 |
| NRAS | 1 | 115258670 | 115258781 | NM_002524.4 |
| ERBB4 | 2 | 212288880 | 212289026 | NM_005235.2 |
| ERBB4 | 2 | 212426628 | 212426813 | NM_005235.2 |
| ERBB4 | 2 | 212488647 | 212488769 | NM_005235.2 |
| ERBB4 | 2 | 212495187 | 212495319 | NM_005235.2 |
| ERBB4 | 2 | 212530048 | 212530202 | NM_005235.2 |
| ERBB4 | 2 | 212576775 | 212576901 | NM_005235.2 |
| ERBB4 | 2 | 212578260 | 212578373 | NM_005235.2 |
| ERBB4 | 2 | 212587118 | 212587259 | NM_005235.2 |
| ERBB4 | 2 | 212589801 | 212589919 | NM_005235.2 |
| ERBB4 | 2 | 212652750 | 212652884 | NM_005235.2 |
| CTNNB1 | 3 | 41266017 | 41266244 | NM_001904.3 |
| PIK3CA | 3 | 178916613 | 178916965 | NM_006218.3 |
| PIK3CA | 3 | 178917477 | 178917687 | NM_006218.3 |
| PIK3CA | 3 | 178919077 | 178919328 | NM_006218.3 |
| PIK3CA | 3 | 178921331 | 178921577 | NM_006218.3 |
| PIK3CA | 3 | 178922290 | 178922376 | NM_006218.3 |
| PIK3CA | 3 | 178927382 | 178927488 | NM_006218.3 |
| PIK3CA | 3 | 178927973 | 178928126 | NM_006218.3 |
| PIK3CA | 3 | 178928218 | 178928353 | NM_006218.3 |
| PIK3CA | 3 | 178935997 | 178936122 | NM_006218.3 |
| PIK3CA | 3 | 178936983 | 178937065 | NM_006218.3 |
| PIK3CA | 3 | 178937358 | 178937523 | NM_006218.3 |
| PIK3CA | 3 | 178937736 | 178937840 | NM_006218.3 |
| PIK3CA | 3 | 178938773 | 178938945 | NM_006218.3 |
| PIK3CA | 3 | 178941868 | 178941975 | NM_006218.3 |
| PIK3CA | 3 | 178942487 | 178942609 | NM_006218.3 |
| PIK3CA | 3 | 178943749 | 178943828 | NM_006218.3 |
| PIK3CA | 3 | 178947059 | 178947230 | NM_006218.3 |
| PIK3CA | 3 | 178947791 | 178947909 | NM_006218.3 |
| PIK3CA | 3 | 178948012 | 178948164 | NM_006218.3 |
| PIK3CA | 3 | 178951881 | 178952152 | NM_006218.3 |
| PDGFRA | 4 | 55129834 | 55130094 | NM_006206.5 |
| PDGFRA | 4 | 55133456 | 55133627 | NM_006206.5 |
| PDGFRA | 4 | 55133719 | 55133908 | NM_006206.5 |
| PDGFRA | 4 | 55138561 | 55138687 | NM_006206.5 |
| PDGFRA | 4 | 55141008 | 55141140 | NM_006206.5 |
| PDGFRA | 4 | 55144529 | 55144682 | NM_006206.5 |
| PDGFRA | 4 | 55146483 | 55146649 | NM_006206.5 |
| PDGFRA | 4 | 55152008 | 55152130 | NM_006206.5 |
| PDGFRA | 4 | 55153597 | 55153708 | NM_006206.5 |
| FBXW7 | 4 | 153244033 | 153244301 | NM_033632.3 |
| FBXW7 | 4 | 153245336 | 153245546 | NM_033632.3 |
| FBXW7 | 4 | 153247158 | 153247383 | NM_033632.3 |
| FBXW7 | 4 | 153249360 | 153249541 | NM_033632.3 |
| FBXW7 | 4 | 153250824 | 153250937 | NM_033632.3 |
| FBXW7 | 4 | 153251884 | 153252020 | NM_033632.3 |
| FBXW7 | 4 | 153253748 | 153253871 | NM_033632.3 |
| FBXW7 | 4 | 153258954 | 153259088 | NM_033632.3 |
| FBXW7 | 4 | 153268082 | 153268223 | NM_033632.3 |
| FBXW7 | 4 | 153332455 | 153332955 | NM_033632.3 |
| APC | 5 | 112043414 | 112043579 | NM_000038.5 |
| APC | 5 | 112074097 | 112074157 | NM_000038.5 |
| APC | 5 | 112090587 | 112090722 | NM_000038.5 |
| APC | 5 | 112102022 | 112102107 | NM_000038.5 |
| APC | 5 | 112102064 | 112102107 | NM_000038.5 |
| APC | 5 | 112102885 | 112103087 | NM_000038.5 |
| APC | 5 | 112111325 | 112111434 | NM_000038.5 |
| APC | 5 | 112116486 | 112116600 | NM_000038.5 |
| APC | 5 | 112128142 | 112128226 | NM_000038.5 |
| APC | 5 | 112136975 | 112137080 | NM_000038.5 |
| APC | 5 | 112151191 | 112151290 | NM_000038.5 |
| APC | 5 | 112151206 | 112151290 | NM_000038.5 |
| APC | 5 | 112154662 | 112155041 | NM_000038.5 |
| APC | 5 | 112154965 | 112155041 | NM_000038.5 |
| APC | 5 | 112157592 | 112157688 | NM_000038.5 |
| APC | 5 | 112159003 | 112159057 | NM_000038.5 |
| APC | 5 | 112162804 | 112162944 | NM_000038.5 |
| APC | 5 | 112163625 | 112163703 | NM_000038.5 |
| APC | 5 | 112164552 | 112164669 | NM_000038.5 |
| APC | 5 | 112170647 | 112170862 | NM_000038.5 |
| APC | 5 | 112173249 | 112179823 | NM_000038.5 |
| EGFR | 7 | 55086971 | 55087058 | NM_005228.4 |
| EGFR | 7 | 55209979 | 55210130 | NM_005228.4 |
| EGFR | 7 | 55210998 | 55211181 | NM_005228.4 |
| EGFR | 7 | 55214299 | 55214433 | NM_005228.4 |
| EGFR | 7 | 55220239 | 55220357 | NM_005228.4 |
| EGFR | 7 | 55221704 | 55221845 | NM_005228.4 |
| EGFR | 7 | 55223523 | 55223639 | NM_005228.4 |
| EGFR | 7 | 55224226 | 55224352 | NM_005228.4 |
| EGFR | 7 | 55225356 | 55225446 | NM_005228.4 |
| EGFR | 7 | 55227832 | 55228031 | NM_005228.4 |
| EGFR | 7 | 55229192 | 55229324 | NM_005228.4 |
| EGFR | 7 | 55232973 | 55233130 | NM_005228.4 |
| EGFR | 7 | 55240676 | 55240817 | NM_005228.4 |
| EGFR | 7 | 55241614 | 55241736 | NM_005228.4 |
| EGFR | 7 | 55259412 | 55259567 | NM_005228.4 |
| EGFR | 7 | 55266410 | 55266556 | NM_005228.4 |
| EGFR | 7 | 55268009 | 55268106 | NM_005228.4 |
| EGFR | 7 | 55268881 | 55269048 | NM_005228.4 |
| MET | 7 | 116339139 | 116340338 | NM_001127500.2 |
| MET | 7 | 116380906 | 116381079 | NM_001127500.2 |
| MET | 7 | 116403104 | 116403322 | NM_001127500.2 |
| MET | 7 | 116411903 | 116412043 | NM_001127500.2 |
| MET | 7 | 116414935 | 116415165 | NM_001127500.2 |
| MET | 7 | 116417443 | 116417523 | NM_001127500.2 |
| MET | 7 | 116423358 | 116423523 | NM_001127500.2 |
| MET | 7 | 116435941 | 116436178 | NM_001127500.2 |
| BRAF | 7 | 140426293 | 140426316 | NM_004333.5 |
| BRAF | 7 | 140434396 | 140434570 | NM_004333.5 |
| BRAF | 7 | 140434416 | 140434570 | NM_004333.5 |
| BRAF | 7 | 140439611 | 140439746 | NM_004333.5 |
| BRAF | 7 | 140449086 | 140449218 | NM_004333.5 |
| BRAF | 7 | 140453074 | 140453193 | NM_004333.5 |
| BRAF | 7 | 140453986 | 140454033 | NM_004333.5 |
| BRAF | 7 | 140476711 | 140476888 | NM_004333.5 |
| BRAF | 7 | 140477790 | 140477875 | NM_004333.5 |
| BRAF | 7 | 140481375 | 140481493 | NM_004333.5 |
| BRAF | 7 | 140482820 | 140482957 | NM_004333.5 |
| BRAF | 7 | 140487347 | 140487384 | NM_004333.5 |
| BRAF | 7 | 140494107 | 140494267 | NM_004333.5 |
| BRAF | 7 | 140500161 | 140500281 | NM_004333.5 |
| BRAF | 7 | 140501211 | 140501360 | NM_004333.5 |
| BRAF | 7 | 140507759 | 140507862 | NM_004333.5 |
| BRAF | 7 | 140508691 | 140508795 | NM_004333.5 |
| BRAF | 7 | 140534408 | 140534672 | NM_004333.5 |
| BRAF | 7 | 140549910 | 140550012 | NM_004333.5 |
| BRAF | 7 | 140624365 | 140624503 | NM_004333.5 |
| PTEN | 10 | 89624227 | 89624305 | NM_000314.6 |
| PTEN | 10 | 89685270 | 89685314 | NM_000314.6 |
| PTEN | 10 | 89692770 | 89693008 | NM_000314.6 |
| PTEN | 10 | 89711875 | 89712016 | NM_000314.6 |
| PTEN | 10 | 89717610 | 89717776 | NM_000314.6 |
| PTEN | 10 | 89720651 | 89720875 | NM_000314.6 |
| ATM | 11 | 108098503 | 108098615 | NM_000051.3 |
| ATM | 11 | 108114680 | 108114845 | NM_000051.3 |
| ATM | 11 | 108115515 | 108115753 | NM_000051.3 |
| ATM | 11 | 108117691 | 108117854 | NM_000051.3 |
| ATM | 11 | 108121428 | 108121799 | NM_000051.3 |
| ATM | 11 | 108123544 | 108123639 | NM_000051.3 |
| ATM | 11 | 108124541 | 108124766 | NM_000051.3 |
| ATM | 11 | 108128208 | 108128333 | NM_000051.3 |
| ATM | 11 | 108137898 | 108138069 | NM_000051.3 |
| ATM | 11 | 108139137 | 108139336 | NM_000051.3 |
| ATM | 11 | 108141978 | 108142133 | NM_000051.3 |
| ATM | 11 | 108151722 | 108151895 | NM_000051.3 |
| ATM | 11 | 108159704 | 108159830 | NM_000051.3 |
| ATM | 11 | 108165654 | 108165786 | NM_000051.3 |
| ATM | 11 | 108172375 | 108172516 | NM_000051.3 |
| ATM | 11 | 108173580 | 108173756 | NM_000051.3 |
| ATM | 11 | 108180887 | 108181042 | NM_000051.3 |
| ATM | 11 | 108186550 | 108186638 | NM_000051.3 |
| ATM | 11 | 108200941 | 108201148 | NM_000051.3 |
| ATM | 11 | 108205696 | 108205836 | NM_000051.3 |
| ATM | 11 | 108206572 | 108206688 | NM_000051.3 |
| ATM | 11 | 108213949 | 108214098 | NM_000051.3 |
| ATM | 11 | 108216470 | 108216635 | NM_000051.3 |
| ATM | 11 | 108218006 | 108218092 | NM_000051.3 |
| ATM | 11 | 108224493 | 108224607 | NM_000051.3 |
| ATM | 11 | 108236052 | 108236235 | NM_000051.3 |
| KRAS | 12 | 25362728 | 25362845 | NM_004985.4 |
| KRAS | 12 | 25368374 | 25368494 | NM_004985.4 |
| KRAS | 12 | 25378547 | 25378707 | NM_004985.4 |
| KRAS | 12 | 25380167 | 25380346 | NM_004985.4 |
| KRAS | 12 | 25398207 | 25398318 | NM_004985.4 |
| BRCA2 | 13 | 32890598 | 32890664 | NM_000059.3 |
| BRCA2 | 13 | 32900636 | 32900750 | NM_000059.3 |
| BRCA2 | 13 | 32906409 | 32907524 | NM_000059.3 |
| BRCA2 | 13 | 32910402 | 32915333 | NM_000059.3 |
| BRCA2 | 13 | 32928998 | 32929425 | NM_000059.3 |
| BRCA2 | 13 | 32930565 | 32930746 | NM_000059.3 |
| BRCA2 | 13 | 32936660 | 32936830 | NM_000059.3 |
| BRCA2 | 13 | 32937316 | 32937670 | NM_000059.3 |
| BRCA2 | 13 | 32944539 | 32944694 | NM_000059.3 |
| BRCA2 | 13 | 32945093 | 32945237 | NM_000059.3 |
| BRCA2 | 13 | 32953887 | 32954050 | NM_000059.3 |
| BRCA2 | 13 | 32972299 | 32972907 | NM_000059.3 |
| RB1 | 13 | 48878049 | 48878185 | NM_000321.2 |
| RB1 | 13 | 48919216 | 48919335 | NM_000321.2 |
| RB1 | 13 | 48934153 | 48934263 | NM_000321.2 |
| RB1 | 13 | 48939030 | 48939107 | NM_000321.2 |
| RB1 | 13 | 48941630 | 48941739 | NM_000321.2 |
| RB1 | 13 | 48942663 | 48942740 | NM_000321.2 |
| RB1 | 13 | 48951054 | 48951170 | NM_000321.2 |
| RB1 | 13 | 48953730 | 48953786 | NM_000321.2 |
| RB1 | 13 | 48955383 | 48955579 | NM_000321.2 |
| RB1 | 13 | 49027129 | 49027247 | NM_000321.2 |
| RB1 | 13 | 49033824 | 49033969 | NM_000321.2 |
| RB1 | 13 | 49037867 | 49037971 | NM_000321.2 |
| RB1 | 13 | 49039134 | 49039247 | NM_000321.2 |
| AKT1 | 14 | 105241413 | 105241544 | NM_005163.2 |
| AKT1 | 14 | 105246425 | 105246553 | NM_005163.2 |
| MAP2K1 | 15 | 66727365 | 66727575 | NM_002755.3 |
| MAP2K1 | 15 | 66729084 | 66729230 | NM_002755.3 |
| TP53 | 17 | 7572926 | 7573008 | NM_000546.5 |
| TP53 | 17 | 7573926 | 7574033 | NM_000546.5 |
| TP53 | 17 | 7576536 | 7576584 | NM_000546.5 |
| TP53 | 17 | 7576624 | 7576657 | NM_000546.5 |
| TP53 | 17 | 7576852 | 7576926 | NM_000546.5 |
| TP53 | 17 | 7577018 | 7577155 | NM_000546.5 |
| TP53 | 17 | 7577498 | 7577608 | NM_000546.5 |
| TP53 | 17 | 7578176 | 7578289 | NM_000546.5 |
| TP53 | 17 | 7578370 | 7578452 | NM_000546.5 |
| TP53 | 17 | 7578370 | 7578533 | NM_000546.5 |
| TP53 | 17 | 7578370 | 7578554 | NM_000546.5 |
| TP53 | 17 | 7579311 | 7579569 | NM_000546.5 |
| TP53 | 17 | 7579311 | 7579590 | NM_000546.5 |
| TP53 | 17 | 7579699 | 7579721 | NM_000546.5 |
| TP53 | 17 | 7579838 | 7579912 | NM_000546.5 |
| NF1 | 17 | 29486028 | 29486111 | NM_001042492.2 |
| NF1 | 17 | 29490204 | 29490394 | NM_001042492.2 |
| NF1 | 17 | 29496909 | 29497015 | NM_001042492.2 |
| NF1 | 17 | 29509526 | 29509683 | NM_001042492.2 |
| NF1 | 17 | 29527440 | 29527613 | NM_001042492.2 |
| NF1 | 17 | 29528429 | 29528503 | NM_001042492.2 |
| NF1 | 17 | 29553453 | 29553702 | NM_001042492.2 |
| NF1 | 17 | 29556043 | 29556483 | NM_001042492.2 |
| NF1 | 17 | 29556853 | 29556992 | NM_001042492.2 |
| NF1 | 17 | 29585362 | 29585520 | NM_001042492.2 |
| NF1 | 17 | 29586050 | 29586147 | NM_001042492.2 |
| NF1 | 17 | 29588729 | 29588875 | NM_001042492.2 |
| NF1 | 17 | 29652838 | 29653270 | NM_001042492.2 |
| NF1 | 17 | 29657314 | 29657516 | NM_001042492.2 |
| NF1 | 17 | 29663653 | 29663932 | NM_001042492.2 |
| NF1 | 17 | 29664386 | 29664600 | NM_001042492.2 |
| NF1 | 17 | 29665043 | 29665157 | NM_001042492.2 |
| NF1 | 17 | 29667523 | 29667663 | NM_001042492.2 |
| NF1 | 17 | 29676138 | 29676269 | NM_001042492.2 |
| NF1 | 17 | 29677201 | 29677336 | NM_001042492.2 |
| NF1 | 17 | 29684287 | 29684387 | NM_001042492.2 |
| NF1 | 17 | 29701031 | 29701173 | NM_001042492.2 |
| ERBB2 | 17 | 37855812 | 37855840 | NM_004448.3 |
| ERBB2 | 17 | 37856491 | 37856564 | NM_004448.3 |
| ERBB2 | 17 | 37863242 | 37863394 | NM_004448.3 |
| ERBB2 | 17 | 37863259 | 37863394 | NM_004448.3 |
| ERBB2 | 17 | 37864573 | 37864787 | NM_004448.3 |
| ERBB2 | 17 | 37865570 | 37865705 | NM_004448.3 |
| ERBB2 | 17 | 37866065 | 37866134 | NM_004448.3 |
| ERBB2 | 17 | 37866338 | 37866454 | NM_004448.3 |
| ERBB2 | 17 | 37866592 | 37866734 | NM_004448.3 |
| ERBB2 | 17 | 37868180 | 37868300 | NM_004448.3 |
| ERBB2 | 17 | 37868574 | 37868701 | NM_004448.3 |
| ERBB2 | 17 | 37871538 | 37871612 | NM_004448.3 |
| ERBB2 | 17 | 37871698 | 37871789 | NM_004448.3 |
| ERBB2 | 17 | 37871992 | 37872192 | NM_004448.3 |
| ERBB2 | 17 | 37872553 | 37872686 | NM_004448.3 |
| ERBB2 | 17 | 37872767 | 37872858 | NM_004448.3 |
| ERBB2 | 17 | 37873572 | 37873733 | NM_004448.3 |
| ERBB2 | 17 | 37873572 | 37873737 | NM_004448.3 |
| ERBB2 | 17 | 37876039 | 37876087 | NM_004448.3 |
| ERBB2 | 17 | 37879571 | 37879710 | NM_004448.3 |
| ERBB2 | 17 | 37879790 | 37879913 | NM_004448.3 |
| ERBB2 | 17 | 37880164 | 37880263 | NM_004448.3 |
| ERBB2 | 17 | 37880978 | 37881164 | NM_004448.3 |
| ERBB2 | 17 | 37881301 | 37881457 | NM_004448.3 |
| ERBB2 | 17 | 37881579 | 37881655 | NM_004448.3 |
| ERBB2 | 17 | 37881959 | 37882106 | NM_004448.3 |
| ERBB2 | 17 | 37882814 | 37882912 | NM_004448.3 |
| ERBB2 | 17 | 37883067 | 37883256 | NM_004448.3 |
| ERBB2 | 17 | 37883547 | 37883800 | NM_004448.3 |
| ERBB2 | 17 | 37883941 | 37883950 | NM_004448.3 |
| ERBB2 | 17 | 37883941 | 37884297 | NM_004448.3 |
| BRCA1 | 17 | 41215350 | 41215390 | NM_007294.3 |
| BRCA1 | 17 | 41219625 | 41219712 | NM_007294.3 |
| BRCA1 | 17 | 41222945 | 41223255 | NM_007294.3 |
| BRCA1 | 17 | 41226348 | 41226538 | NM_007294.3 |
| BRCA1 | 17 | 41228505 | 41228631 | NM_007294.3 |
| BRCA1 | 17 | 41243452 | 41246877 | NM_007294.3 |
| BRCA1 | 17 | 41247863 | 41247939 | NM_007294.3 |
| SMAD4 | 18 | 48573417 | 48573665 | NM_005359.5 |
| SMAD4 | 18 | 48575056 | 48575230 | NM_005359.5 |
| SMAD4 | 18 | 48575665 | 48575694 | NM_005359.5 |
| SMAD4 | 18 | 48581151 | 48581363 | NM_005359.5 |
| SMAD4 | 18 | 48584495 | 48584614 | NM_005359.5 |
| SMAD4 | 18 | 48586236 | 48586286 | NM_005359.5 |
| SMAD4 | 18 | 48591793 | 48591976 | NM_005359.5 |
| SMAD4 | 18 | 48593389 | 48593557 | NM_005359.5 |
| SMAD4 | 18 | 48603008 | 48603146 | NM_005359.5 |
| SMAD4 | 18 | 48604626 | 48604837 | NM_005359.5 |
| GNAS | 20 | 57484405 | 57484478 | NM_000516.5 |
| GNAS | 20 | 57484576 | 57484634 | NM_000516.5 |

### V. NUCLEIC ACID MANIPULATION

A. Repair of Fragmented DNA

There are two main types of DNA end damage that result in DNA ends that are not competent for ligation: ends that are not blunt; and ends that lack a phosphate at a 5′-end and/or have a phosphate at a 3′-end.

The first type of damage can be repaired by the concerted action of a DNA polymerase that extends recessed ends in the presence of deoxynucleotide triphosphates (dNTPs) or a 3′ exonuclease that trims protruding 3′ ends to produce blunt ends. The most commonly used enzyme for this type of repair is T4Pol, which has both DNA polymerase and DNA 3′ exonuclease activities residing on the same protein. However, use of T4Pol may result in over-trimming, thus producing one or two base recessed ends that are not competent for ligation. Klenow has the same enzymatic activities as T4Pol but much weaker 3′ exonuclease than its counterpart. This property makes it a useful supplement to T4Pol for reducing the risk of over-trimming and making the blunt-end reaction more efficient.

The second type of damage can be repaired by enzymatic activities that transfer phosphates to the 5′ termini of DNA and remove phosphates from the 3′ termini of DNA, such as 3′ phosphatases and/or 3′ exonucleases that are not inhibited by the presence of 3′ phosphate, such as, for example, PNK. PNK transfers phosphate from deoxynucleotide triphosphates to the 5′ termini of DNA in a reversible reaction that depends on the concentration of dNTPs, i.e., high dNTP concentrations shift the equilibrium toward transfer to DNA while high concentrations of diphosphates stimulates the reverse reaction. PNK also has an intrinsic 3′-phosphatase activity that removes phosphate from the 3′ termini of DNA but this activity is often insufficient to achieve complete repair.

As provided herein, one example of a multifunctional enzyme that improves the efficiency DNA end-repair is ExoIII. ExoIII catalyzes the stepwise removal of mononucleotides from 3′-hydroxyl termini of double-stranded DNA. ExoIII's 3′-phosphatase activity removes 3′-terminal phosphates, thereby generating 3′-OH groups. It also has class II apurinic/apyrimidinic endonuclease activity, which facilitates hydrolysis of the abasic sites to produce 3′-OH and 5′-PO₄ ends.

For example, a composition is provided comprising T4 DNA Polymerase (T4Pol), T4 Polynucleotide Kinase (T4PNK), ExoIII, and the Large Klenow fragment of E. coli DNA Polymerase I (Klenow). Use of such a composition in DNA end-repair reactions results in improved and robust end-repair, over a large DNA input range, for the purposes of cloning, amplification, and Next Generation Sequencing (NGS) library preparation.

Those skilled in the art will realize that in the case that the target nucleic acid lacks a 3′-OH and/or has a naturally blocked, non-extendable 3′ terminus (such as, for example, a 3′ terminal phosphate, a 2′,3′-cyclic phosphate, a 2′-O-methyl group, a base modification, a backbone sugar or phosphate modification, etc.), the blocked 3′ terminus can be repaired or cleaved to expose a 3′-OH by enzymatic treatment to remove the blocking group prior to proceeding with the methods. In some aspects, repair of the 3′ ends of a target nucleic acid molecule may be performed by a polymerase (e.g., T4 DNA polymerase, Klenow fragment), a kinase (e.g., T4 polynucleotide kinase), a phosphatase (e.g., alkaline calf intestinal phosphatase), a 3′ exonuclease (e.g., exonuclease I, exonuclease III), and/or a restriction endonuclease. In this method, input DNA may be simultaneously fragmented, repaired, and ligated to adaptors. This is accomplished by incubating the input DNA with a polymerase (e.g., T4 DNA polymerase, Klenow fragment), a kinase (e.g., T4 polynucleotide kinase), a phosphatase (e.g., alkaline calf intestinal phosphatase), a 3′ exonuclease (e.g., exonuclease I, exonuclease III), a DNA ligase, and ligation adaptors. In other aspects, these reactions can also be performed sequentially such that the fragments under repair and then repaired fragments are incubated with a DNA ligase and ligation adaptors.

B. Amplification

A number of template-dependent processes are available to amplify the nucleic acids present in a given template sample. One of the best known amplification methods is the polymerase chain reaction (referred to as PCR™) which is described in detail in U.S. Pat. Nos. 4,683,195, 4,683,202, and 4,800,159, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTP's) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus, the template region between the two primers is amplified exponentially, rather than linearly.

A second barcode, such as a sample barcode, may be added to the target nucleic acid molecules during amplification. One method (e.g., described in PCT/US2013/068468, incorporated herein by reference) involves annealing a primer to the first barcoded nucleic acid molecule, the primer including a first portion complementary to the first barcoded nucleic acid molecule and a second portion including a second barcode; and extending the annealed primer to form a dual barcoded nucleic acid molecule, the dual barcoded nucleic acid molecule including the second barcode, the first barcode, and at least a portion of the nucleic acid molecule. Thus, the primer may include a 3′ portion and a 5′ portion, where the 3′ portion may anneal to a portion of the first barcode and the 5′ portion comprises the second barcode.

C. Sequencing

Methods are also provided for the sequencing of the library of adaptor-linked fragments. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit. In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.).

In particular aspects, the sequencing technologies used in the methods of the present disclosure include the HiSeg™ system (e.g., HiSeg™ 2000 and HiSeg™ 1000), the NextSeg™ 500, and the MiSeg™ system from Illumina, Inc. The HiSeg™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeg™ system uses TruSeq™, Illumina's reversible terminator-based sequencing-by-synthesis.

Another example of a DNA sequencing technique that can be used in the methods of the present disclosure is 454 sequencing (Roche). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2009). Complete genomics' CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

### VI. ANALYSIS OF NGS READS

The SAMVAR tool was developed to accurately identify variants present at low allelic frequencies. SAMVAR is a fully automated next generation sequencing data analysis pipeline that integrates DNA template specific dual molecular barcodes, derives a consensus sequence from reads sharing the same molecular barcode, retrieves variants present in those consensus reads, corrects sequencing artifacts, performs annotation of accurate variants, and generates final variant report and variant call format (VCF) files that incorporate all variant associated information.

Consensus sequence derivation. The first 14-nucleotide molecular barcode information from the sequencing reads in forward FASTQ file and the corresponding reads in reverse FASTQ file were combined with SAMVAR. The resulting 28-nucleotide molecular barcode was used to replace the original index of the sequencing read in forward file and the corresponding index of the sequencing read in reverse file. Sequencing reads that shared the same molecular barcode index were referred to as a family. The reads that belonged to a family were grouped together, and from these reads a single consensus sequence (SCS) was derived. The following guidelines were implemented while deriving consensus nucleotide bases in SCS reads.

1) For a chosen position, if the same nucleotide was present across all the reads of the family, it was chosen to represent that position in the consensus read. An average value of quality scores of nucleotide bases from which this consensus base derived was used as a new quality score of the consensus base.

2) For those positions having more than one nucleotide type across all the reads of the family, the majority base was chosen as a consensus base. The quality score of this ambiguous consensus base was adjusted to zero.

3) For a chosen position, if more than one nucleotide bases were observed and the majority base could not be determined, the base with highest quality score was chosen as a consensus base, and the quality score of the consensus base at this ambiguous position was modified to zero.

4) For a chosen position, if the majority base could not be determined and the quality scores of the involved bases remain same, the ambiguity was represented in the consensus read with the letter ‘N’. The quality score of ‘N’ base in the consensus read was adjusted to zero.

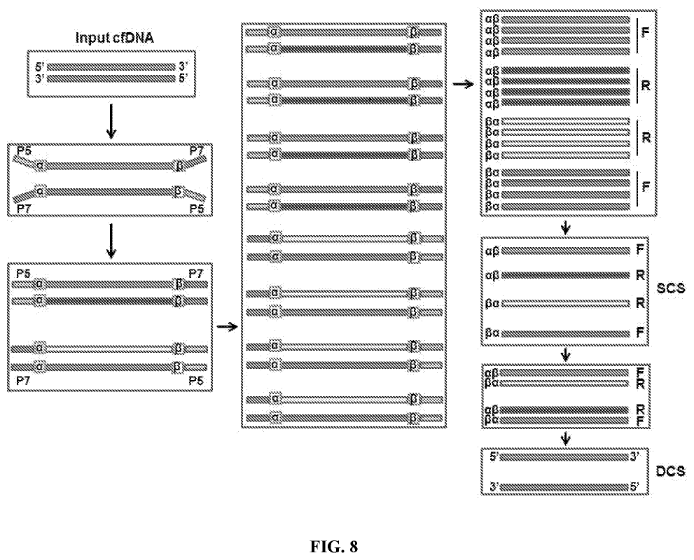

Single consensus sequences were derived independently for the families in forward and reverse sequencing files, and these derived reads were used as templates for subsequently generating double consensus sequences (DCS), and also for improving accurate variants detection from SCS reads. Asymmetric adaptors used in this study supposedly yield top template strand generated sequences with αβ orientation of molecular barcode index (the first 14-nucleotide sequence of molecular barcode is referred to as ‘α’ and the second half of the 14-nucleotide sequence of molecular barcode is referred to as ‘β’), and the bottom template strand generated sequences with βα orientation of molecular barcode index (FIG. 8). SCS read having an αβ orientated molecular barcode index in the forward file was grouped with a SCS read that had a βα orientated molecular barcode index in the reverse file, and DCS read was derived and written to a forward file by assigning the molecular barcode index in αβ orientation. Then an SCS read having the same αβ orientated molecular barcode index in the reverse file was grouped with an SCS read that had a βα orientated molecular barcode index in the forward file; DCS read was derived and written to a reverse file by keeping the molecular barcode index in αβ orientation. The same criterion utilized for deriving consensus bases and their quality scores in SCS reads were implemented for deriving consensus bases in DCS reads. Under circumstances when an SCS read with αβ oriented molecular barcode index either in forward or reverse file did not contain a mate SCS read with βα oriented molecular barcode index in reverse or forward file, respectively, those SCS reads were omitted while creating DCS read files.

In order to improve accuracy of nucleotide bases with in SCS reads further, we implemented mate matching approach and adjusted the quality scores to zero at unmatched positions in following manner. A αβ oriented index containing SCS read from forward file was grouped with βα oriented index containing SCS read from reverse file, and αβ oriented index containing SCS read from reverse file was grouped with βα oriented index containing SCS read from forward file. At positions where ambiguity is encountered, the quality scores in both SCS reads were adjusted to zero and returned to files from where these reads were taken. With this approach the accuracy of nucleotide bases in SCS reads was improved similar to those in the DCS reads.

Variant identification. SCS reads that were derived from families containing two or more reads were used for variant identification, as errors accrued in one-read families cannot be corrected. However, SCS reads from a single read family were retained only under circumstances where corresponding SCS read mate with either αβ/βα orientation was available for correcting sequencing artifacts. Reads were aligned to human reference genome (hg19) with Bowtie2 using sensitive mode and local alignment settings in which the unaligned nucleotides from the 5′ and 3′ ends of the sequencing reads were soft clipped. Bowtie2 produced sam files were converted to bam files, and further these bam files were sorted, indexed using Samtools version 1.8. Position specific variants were determined from the sorted and indexed bam files using Bam-readcount tool. The nucleotide positions for which the base quality was adjusted to zero during consensus sequence derivation were ignored categorically while determining the variants through Bam-readcount analysis. Following the same approach, DCS reads were also aligned and variants were identified. Bam files were converted into BED files, and target regions sequencing coverage were determined using Bedtools version 2.27.1.

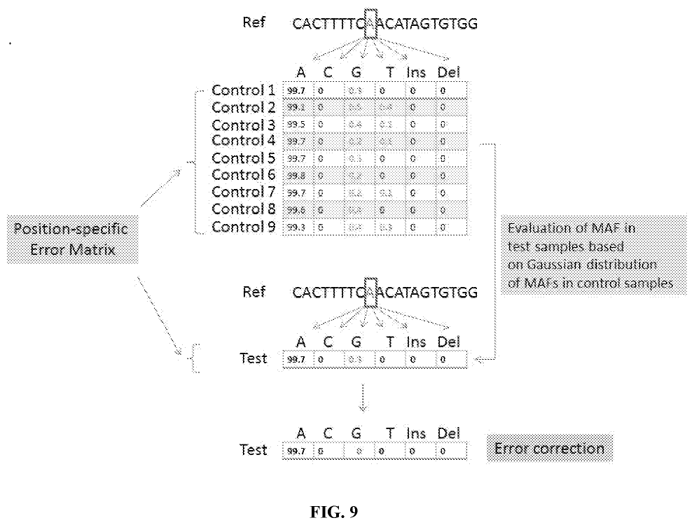

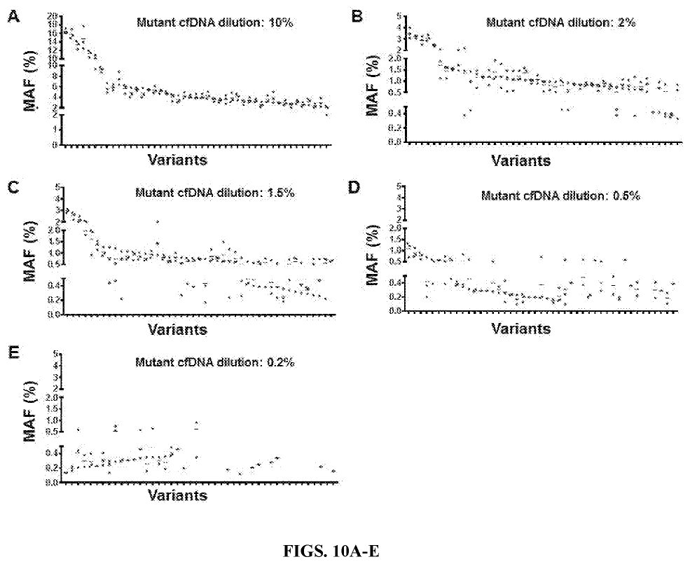

Background error elimination. Bam-readcount output files were configured and the background error correction was carried out with SAMVAR. In order to perform error correction, nine cfDNA libraries that were prepared from healthy donor plasma specimens were sequenced. Variants occurring at a frequency less than 20% were considered to be background error, and a position-specific error model was created (FIG. 9). Variant allele frequencies in the test samples were evaluated based on Gaussian distribution modeled variant frequencies in control samples. If the variant frequencies were determined to be in error, the values were adjusted to zero to eliminate the error. Additional conditions that were applied for correcting the variant frequency errors in test samples were as follows: for chromosome positions at which the variant was occurring, the variant allele count was less than 4; the read balance ratio was less than 0.1; the average quality score of detected variant base was less than 30; the ratio of variant frequency in test sample to control value from the Gaussian distribution model was less than two-fold different.

Variant annotation. Error corrected variants were filtered and true variants were identified with SAMVAR. An input file for variant annotation was developed using SAMVAR, and annotation of variants was performed with Annoavar version 2018 Apr. 16. Finally, a variant report with annotated variants information and VCF 4.2 version file were generated.

### VII. KITS AND DIAGNOSTICS

Kits are envisioned containing diagnostic agents, therapeutic agents, and/or other therapeutic and delivery agents. The kit may comprise reagents capable of use in determining the variant allele frequency of at least a portion of the genomic loci listed in Table 1. For example, reagents of the kit may include at least 20, 25, 30, 35, 40, 45, 50, 55, 60, 65, 70, 75, 80, 85, 90, 95, or 100 RNA biats, as well as reagents to prepare the target nucleic acids for analysis. The kit may also comprise a suitable container means, which is a container that will not react with components of the kit, such as an eppendorf tube, a syringe, a bottle, or a tube. The container may be made from sterilizable materials such as plastic or glass. The kit may further include an instruction sheet that outlines the procedural steps of the methods, such as the same procedures as described herein or are otherwise known to those of ordinary skill.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Panel design. Sequencing data from a cohort of 2,906 colorectal cancer patients was examined and, using this information, a panel was designed that spans 78.81 Kb (referred to as CRC23; Table 1) and covers 85% of the most frequently mutated targets in this cohort. All coding exons of TP53, APC, KRAS, NRAS, BRAF, PIK3CA, and ERBB2 and hotspot coding exons from 16 other genes were covered with this panel.

Samples. Blood specimens from 32 patients with colorectal adenocarcinoma were collected after informed consent. All samples used in this study were from patients with stage 4 disease. Blood samples were collected in Vacutainer tubes coated with K2EDTA, and plasma was separated within 2-4 hours of specimen collection by centrifuging at 400×g for 10 minutes and stored at −80° C. Plasma from healthy donors was obtained from the institutional blood bank under an approved IRB protocol. Culture supernatants from the cell lines MOLT-4, HT-29, DLD-1, and OCI-AML3 were centrifuged at 400×g for 10 minutes and stored at −80° C.

Isolation of cfDNA. Frozen plasma samples or cell culture supernatants were thawed in a room temperature water bath and centrifuged at 1600×g for 10 minutes to remove precipitated debris. From the clear supernatants, cfDNA was isolated either by a manual extraction method or by an automated extraction method on QIAsymphony following guidelines provided by the vendor (Qiagen, Germantown, Md.). The cfDNA that was extracted by manual methods often contained high-molecular-weight genomic DNA. Therefore, on these cfDNA samples size selection was performed, contaminating genomic DNA was removed, and 166-bp fragments of cfDNA were retained. Briefly, 50 μl of cfDNA was mixed with 35 μl of SPRIselect beads, incubated at room temperature for 15 minutes, and further incubated on a magnetic plate for 10 minutes. Clear supernatant was collected, and beads bound to the genomic DNA were discarded. Supernatant was mixed with 65 μl of SPRIselect beads, incubated at room temperature for 15 minutes, and further incubated on a magnetic plate for 10 minutes. Then, supernatants were discarded, and beads were washed twice with 200 μl of 85% alcohol and air dried at room temperature for 10 minutes; cfDNA was eluted in 56 μl of 10 mM Tris-Cl, pH 8.0, and stored at −20° C.

Preparation of sequencing library. Libraries were prepared using the NEBNext ultra II DNA library prep kit (New England Biolabs, Ipswich, Mass.) with the following modifications. Five to 30 nanograms of cfDNA in 50 μl were mixed with 7 μl of end-repair reaction buffer and 3 μl of end-repair enzyme mix and incubated at 20° C. for 45 minutes. Following incubation, enzyme components were inactivated by heating at 65° C. for 30 minutes. For each 30 μl of end-repair reaction volume, 2.5 μl of 30 ng/μl adaptor, 30 μl of ligation enzyme mix, and 1 μl of ligation enhancer were added and incubated at 20° C. for 30 minutes. Then, 3 μl of USER enzyme mix was added and incubated at 37° C. for 20 minutes, and adaptor ligated cfDNA was purified using SPRIselect beads. Briefly, 60 μl of SPRI beads were mixed with 66.5 μl of library reaction components and incubated at room temperature for 5 minutes and on a magnetic plate for an additional 10 minutes. Bead-free supernatants were removed, leaving approximately 15 μl of solution to prevent the loss of library bound beads. Beads were washed twice with 200 μl of 85% alcohol and air dried at room temperature for 10 minutes, and the library was eluted in 40 μl of 10 mM Tris-Cl, pH 8.0.

Post-library preparation amplification. Adaptor-ligated cfDNA templates were amplified in a polymerase chain reaction (PCR) prior to enriching target regions through hybridization capture. Briefly, reactions were assembled in 100 μl by mixing 50 μl of NEBNext ultra II Q5 master mix, 14 μl of 10 μM forward and reverse primer mix, and 36 μl of adapter ligated cfDNA. PCR amplification was performed in three stages: during the first stage, initial denaturation was performed at 98° C. for 30 seconds; during the second stage, sequential incubations were performed at 98° C. for 10 seconds, 85° C. for 1 second, and 68° C. for 6 minutes for a total of 10 cycles; during the third stage, the final extension was conducted at 68° C. for 5 minutes, and samples were held finally at 4° C. (In the second stage, during the 85° C. to 68° C. transition, a ramp rate of 0.2° C./second was used.) PCR amplification products were purified using SPRIselect beads; 90 μl of beads were mixed with 1000 of PCR products and purification was performed following the steps described earlier.

Target regions hybridization capture. As described earlier, after end repair the final volume of 60 μl was divided into two tubes and subsequent steps were performed independently on each tube. The resultant amplification reactions (n=2) from the same sample were pooled after purification, and the DNA library concentration was quantified with Qubit (Thermofisher Scientific, Waltham, Mass.). DNA blocker mix was prepared by adding 2.5 μl of 10 μg/μl salmon sperm DNA, 2.5 μl of 1 μg/μl cot-1 DNA, and 0.6 μl of 1000 μM adaptor blockers. A DNA library of 500-1000 ng was concentrated into 5.6 μl by vacuum centrifugation, mixed with 3.4 μl of DNA blocker mix, and incubated at 95° C. for 5 minutes and 65° C. for 10 minutes. RNA baits hybridization mix was prepared by adding 13 μl of hybridization buffer (6.63 μl of 20×SSPE, 0.27 μl of 0.5 M EDTA, 2.65 μl of 50×Denhardt's solution, and 3.45 μl of 0.76% SDS), 2 μl of RNase blocking solution (0.5 μl of SUPERase In RNase inhibitor (20 U/μl) and 1.5 μl of nuclease-free water), and 5 μl of enrichment baits solution (1.5 ng/μl); this mix was incubated at 65° C. for 5 minutes. At the end of the incubation period, 20 μl of enrichment baits capture mix was transferred to the DNA library and blocker mix, and the incubation was continued at 65° C. for 16 hours.

Streptavidin T1 beads were prepared for binding by washing 50 μl of beads with 200 μl of binding buffer (10 ml of 5 M NaCl, 0.5 ml of 1 M Tris-Cl, pH 7.5, 0.1 ml of 0.5 M EDTA, and 39.4 ml of nuclease-free water) three times, and beads were finally re-suspended in 200 μl of binding buffer. At the end of 16 hours of incubation, approximately 26 μl of hybridization capture mixture was added to 200 μl of streptavidin beads and incubated on a mixer at 1600 rpm for 1 hour. Subsequently, beads were washed with wash1 buffer (2.5 ml of 20×SSC, 0.5 ml of 10% SDS, and 47 ml of nuclease-free water) at room temperature for 15 minutes, and a total of four washes was performed with wash2 buffer (0.25 ml of 20×SSC, 0.5 ml of 10% SDS, and 49.25 ml of nuclease-free water) at 65° C. incubation for 10 minutes during each wash. Beads were re-suspended in 30 μl of 0.1 N NaOH and incubated at room temperature for 10 minutes to elute the target DNA from streptavidin beads. Elute was neutralized with 30 μl of 1 M Tris-Cl, pH 7.5; DNA was purified with 120 μl of SPRIselect beads following the steps described earlier; and DNA was eluted in 44 μl of 10 mM Tris-Cl, pH 8.0.

Post-hybridization capture amplification. Enriched DNA targets were amplified in PCR. Briefly, reactions were assembled in 100 μl by mixing 50 μl of NEBNext ultra II Q5 master mix, 10 μl of 10 μM Illumina index primer mix, and 40 μl of DNA elute from hybridization capture. PCR amplification was performed in four stages: during the first stage, initial denaturation was performed at 98° C. for 30 seconds; during the second stage, sequential incubations were performed at 98° C. for 10 seconds, 85° C. for 1 second, and 68° C. for 6 minutes for a total of 10 cycles; during the third stage, an additional four cycles of amplification were performed at 98° C. for 10 seconds, 85° C. for 1 second, and 68° C. for 90 seconds; during the fourth stage, the final extension was conducted at 68° C. for 5 minutes, and samples were held finally at 4° C. During the 85° C. to 68° C. transitions, a ramp rate of 0.2° C./second was applied. PCR amplification products were purified using SPRIselect beads following the steps described earlier, and DNA libraries were eluted in 100 μl of 10 mM Tris-Cl, pH 8.0. These DNA libraries were double size selected with 0.56×/0.85×SPRI beads as described earlier and finally eluted in 40 μl of 10 mM Tris-Cl, pH 8.0.

Sequencing. Libraries were quantified on the 4200 TapeStation system (Agilent Technologies, Santa Clara, Calif.); typically, the library concentrations were in the range of 2-5 nM. A total of 21 indexed libraries (including a positive control library and a negative control library) were pooled, denatured, and diluted to a final concentration of 2.2 pM following guidelines provided by the vendor (Illumina, San Diego, Calif.). Libraries that were created by diluting a mutant cfDNA pool (MOLT-4, HT-29, and DLD-1) into a control cfDNA (OCI-AML3) at 1% frequency were used as a positive control, and a library from healthy donor cfDNA was used as negative control in each sequencing run. Pooled libraries were mixed with PhiX library at a 4:1 ratio and sequenced on Nextseq550 using a high output flow cell (Illumina).

### Example 2—Optimization of Enrichment Baits Improves On-Target Hybridization Capture Efficiency

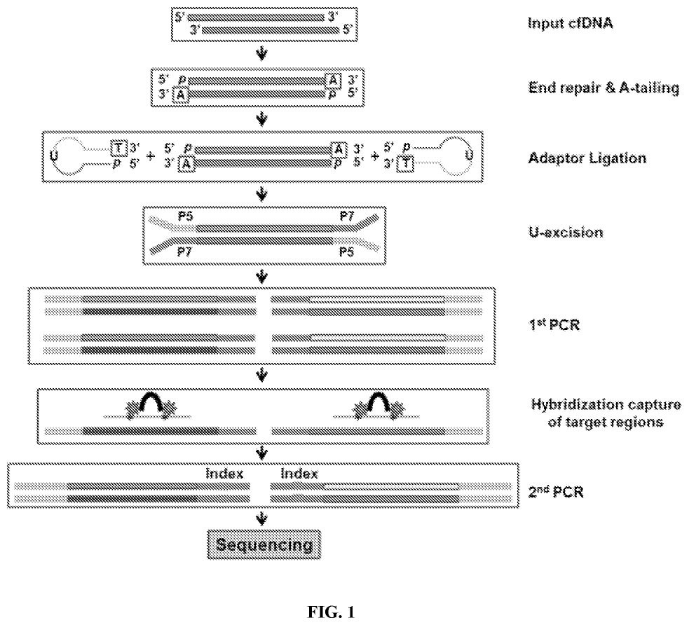

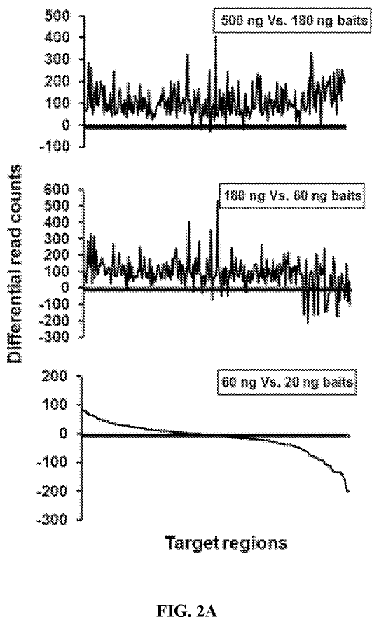

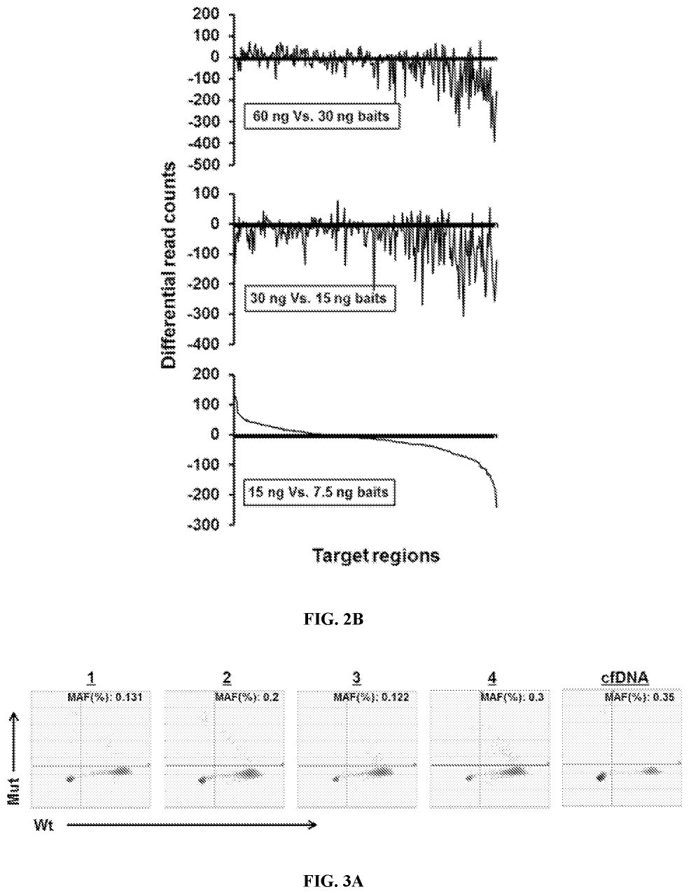

Each sequencing ready library was prepared in four stages, with the stages essentially being library preparation, post-library amplification, hybridization capture of target regions of interest, and post-hybridization capture amplification (FIG. 1). The steps involved in each of these four stages were optimized in order to preserve the initial variant allele frequencies throughout all stages of library generation. It was hypothesized that the quality and quantities of the final sequencing library are critically influenced by proportions of DNA target and enrichment baits used during hybridization capture of target molecules of interest. Therefore, the quantities of enrichment baits critical to the assay performance were evaluated by performing hybridization capture with various quantities of enrichment baits (FIGS. 2A-B, Tables 1& 2). After sequencing the libraries from these enrichments, it was found that 180 ng of baits compared with 500 ng of baits, and 60 ng of baits compared with 180 ng of baits could yield higher sequencing coverage of target regions. This high sequencing coverage was also accompanied by higher on-target percentages (Table 1). Furthermore, while 20-ng baits compared with 60-ng baits did not improve the sequencing coverage strikingly, they could yield a higher on-target rate. These observations were similar when baits were serially diluted from 60 ng to 7.5 ng and used in hybridization capture (FIG. 2B, Table 2). During the panel design, 2× tiled probe sequences were created, indicating that each 60-bp target region was covered by overlapping two-probe sequences. For this reason, each half of the probe at the boundaries of the target region will be involved in enriching target flanking regions. To accommodate target flanking regions in our on-target rate calculations, an additional 200-bp flanking region was padded on to the target sequences, and this adjustment yielded higher on-target percentages (Tables 1& 2). Greater than 80% on-target enrichment was observed with 7.5 ng of enrichment baits, and this concentration of enrichment baits was used thereafter for all hybridizations. These findings suggest that on-target enrichment is critically influenced by the quantity of enrichment baits utilized during the hybridization capture stage.

TABLE 1
Enrichment baits concentration optimization (20 ng-500 ng).
On-target rate was calculated by dividing the mapped read
coverage of target regions with total mapped reads coverage.

| Quantity of | | On target capture (%) |
|---|---|---|
| Baits in | | (with 200bp |
| hybridization | On target | additional |
| capture | capture (%) | padding) |
| 500 ng | 30.25 | 38.21 |
| 180 ng | 46.84 | 58.04 |
| 60 ng | 63.39 | 76.24 |
| 20 ng | 66.73 | 79.43 |

TABLE 2
Enrichment baits concentration optimization (7.5 ng-60 ng).
On-target rate was calculated by dividing the mapped read
coverage of target regions with total mapped reads coverage.

| Quantity of | | On target capture (%) |
|---|---|---|
| Baits in | | (with 200bp |
| hybridization | On target | additional |
| capture | capture (%) | padding) |
| 60 ng | 60.76 | 74.35 |
| 30 ng | 66.33 | 79.67 |
| 15 ng | 63.98 | 75.93 |
| 7.5 ng | 71.90 | 84.37 |

### Example 3—Steps Critical to Incorporating Low Frequency Variant Encoding cfDNA Templates into a Sequencing Library

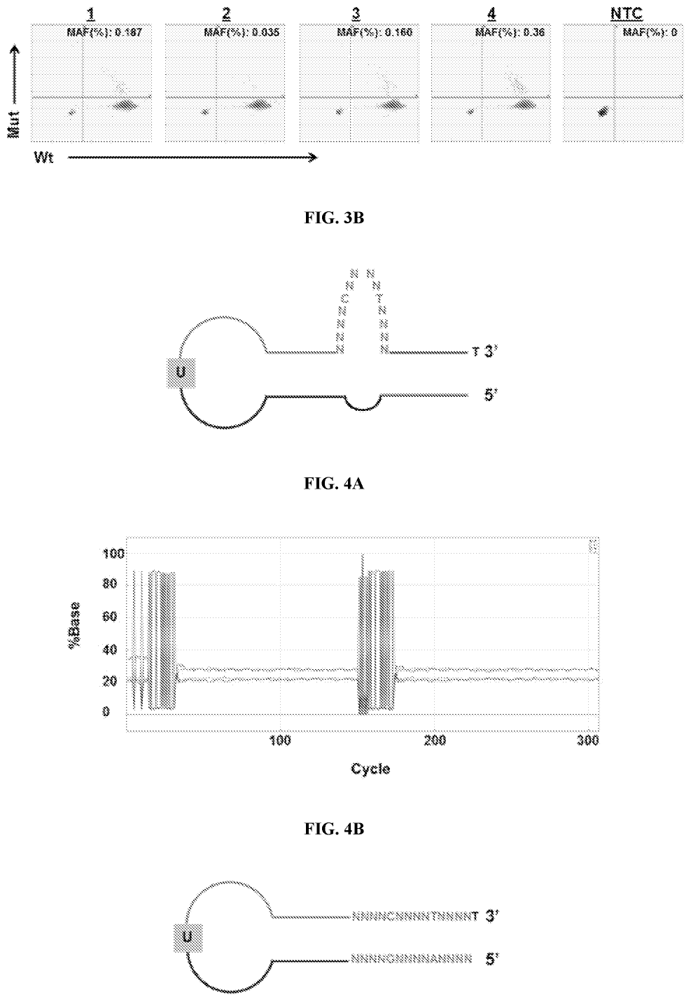

Identifying the conditions that maximize incorporation of cfDNA templates into libraries is critical for ultra-sensitive detection of true variants present at low allelic frequencies. The cfDNA pool was created by mixing cfDNA harvested from the MOLT-4, HT-29, and DLD-1 cell lines (mutant) and the OCI-AML3 line (control, negative for the variants present in the mutant pool) at 2%, 1%, 0.2%, and 96.8% proportions, respectively. In this cfDNA mix, the expected BRAF V600E variant allelic frequency was 0.5% (Table 3). Using this cfDNA mix, the libraries were generated under various conditions (Table 4) and the pre-enrichment and post-enrichment libraries were evaluated through droplet digital PCR-based detection of the BRAF V600E variant (FIGS. 3A, 3B). In comparison with the BRAF V600E variant frequency in the original cfDNA template pool, reduced frequencies were found in libraries that were prepared under conventional conditions (FIGS. 3A, 3B panel 1). It was hypothesized that end repair reaction mixture carryover into the ligation mixture might be hampering ligation efficiency and could be the cause of reduced variant allelic frequencies. To test this hypothesis, an additional purification step was integrated after the end repair reaction (Table 4). The libraries that were prepared following this additional purification also yielded reduced frequencies of the BRAF V600E variant (FIGS. 3A, 3B; panel 2) compared with its frequency in the original template pool used for library construction. To mitigate the inhibitory effect of end repair components on the ligation mixture, the dilution of end repair reaction components prior to mixing with the ligation mixture was evaluated; this modification was also accompanied by reduction in the BRAF V600E variant frequency (FIGS. 3A, 3B; panel 3). Another variation that was tested was adding half of the end repair reaction final components to the ligation mixture. Surprisingly, concordant variant allele frequencies were observed between the original cfDNA template and pre- and post-enrichment libraries that were prepared following this modification (FIGS. 3A, 3B; panel 4).

TABLE 3
Preparation of a mutant cfDNA pool for library
generation. The cfDNA from MOLT-4, HT-29,
DLD-1 (mutant), and OCI-AML3 (control) cell
lines were mixed at 2%, 1%, 0.2%, and 96.8%
frequencies. Note that in this mutant pool the
expected frequency of BRAF V600E is 0.5%.

| | | cfDNA | Expected BRAF |
|---|---|---|---|
| | | Dilution (%) | V600E MAE (%) |
| Mutant | MOLT-4 | 2 | 0.5 |
| | HT-29 | 1 | |
| | DLD-1 | 0.2 | |
| Control | OCI-AML3 | 96.8 | |

TABLE 4
An outline of four different library preparation workflows evaluated in this study.
Note that in workflows 3 and 4, after end repair reaction, volume was split into two tubes and
processed independently up to the first PCR amplification post purification step.

| | 1 | 2 | 3 | 4 | | |
|---|---|---|---|---|---|---|
| Input cfDNA quantity (ng) | 30 | 30 | 30 | 30 | | |
| End-repair reaction volume (ul) | 60 | 60 | 60 | 60 | | |
| Post end-repair purification | − | + | − | − | | |
| | 1 | 2 | 3A | 3B | 4A | 4B |
| End-repair carry over volume (ul) | 60 | 60 | 30 | 30 | 30 | 30 |
| 10 mM Tris-Cl (ul) | | | 30 | 30 | | |
| Ligation mix volume (ul) | 30 | 30 | 30 | 30 | 30 | 30 |
| Post ligation purification | + | + | + | + | + | + |
| 1^{st} PCR | + | + | + | + | + | + |
| Post PCR purification | + | + | + | + | + | + |

| | | | | |
|---|---|---|---|---|
| Hybridization capture: DNA (ng) | 1500 | 1500 | 1500 | 1500 |
| Hybridization capture: baits (ng) | 7.5 | 7.5 | 7.5 | 7.5 |
| 2^{nd} PCR | + | + | + | + |
| Post PCR purification | + | + | + | + |

### Example 4—Structure of Adaptors Influences Acquisition of Single or Dual Molecular Barcodes

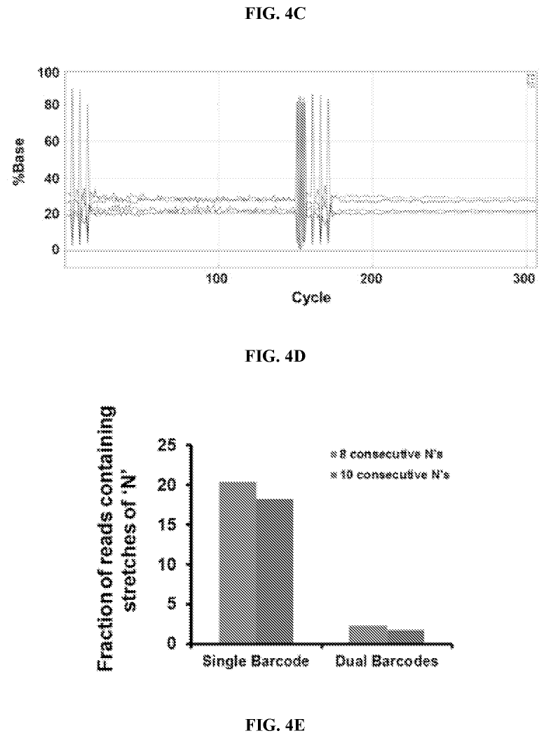

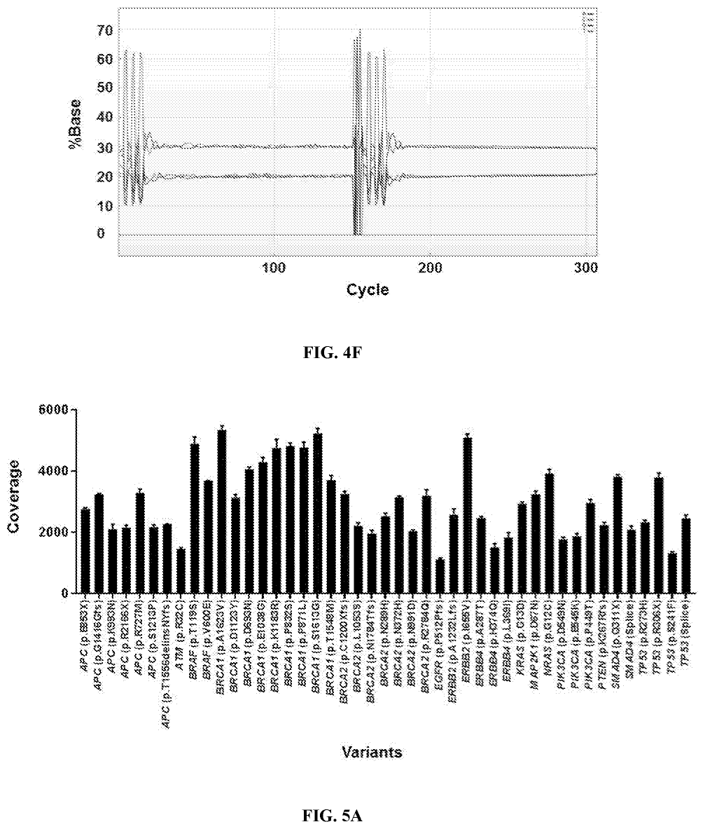

The structure of the molecular barcode sequence-containing adaptors facilitates incorporation of single or dual barcode information into the sequencing reads. In this study, two versions of adaptors were evaluated. The first version yields one individual barcode at the 5′ end of the sequencing read (referred to as single molecular barcode adaptor) (FIGS. 4A, 4B). The second version yields dual barcodes, positioned at the 5′ and 3′ ends of the sequencing read (referred to as dual molecular barcode adaptor) (FIGS. 4C, 4D). Structure of these adaptors was further evident from the depictions of the sequence analysis viewer (FIGS. 4B, 4D). While synthesizing the adaptors, ‘C’ and ‘T’ were positioned at the 5th and 10th positions of barcode sequence as place holders; these nucleotides were common to all barcodes. In the case of a single molecular barcode adaptor, the first two distinct peaks noticed in the sequence analysis viewer represent ‘C’ and ‘T’ nucleotides present at the 5th and 10th positions. Following the molecular barcode sequence, a universal 18-nucleotide sequence was present. These nucleotides were noticed at greater than 80% frequency, confirming the presence of a universal sequence (FIG. 4B). In the case of dual molecular barcode adaptors, in addition to ‘C’ and ‘T’ at the 5th and 10th positions of the barcode, a ‘T’ nucleotide, which was added through T-tailing reaction at the 15th position, was apparent. The presence of these three peaks at the beginning of the forward and reverse reads suggests the incorporation of two molecular barcode sequences into each sequencing read (FIG. 4D).

While processing sequencing data, reads that shared the same molecular barcode tag were grouped together, and a consensus sequence was derived. For positions that had 100% concordantly matching nucleotides across all the reads sharing similar molecular barcode tags, those concordant nucleotides were chosen in the consensus sequence. If the nucleotides were not 100% concordant, the ambiguity at those positions was indicated by ‘N’ in the consensus sequence. The single barcode adaptors compared with dual barcode adaptors yielded an approximately 6-fold higher fraction of the consensus reads containing 8- to 10-nucleotide stretches of ‘N’ (FIG. 4E). These findings suggest the possibility of diverse templates ligating to the same molecular barcode sequence containing adaptors, hence the occurrence of stretches of ‘N’ in the consensus sequence. Furthermore, the unique molecular barcode tag to cfDNA template ratio was higher in the case of dual molecular barcode adaptors compared with single molecular barcode adaptors, suggesting the possibility of diverse templates sharing the same molecular barcode tag if single molecular barcode adaptors were used (Table 5). On the basis of these observations, in all the subsequent experiments, dual molecular barcode adaptors were used to facilitate performing duplex sequencing (Schmitt et al., 2012; Kennedy et al., 2014; Stoler et al., 2016) (FIG. 8).

TABLE 5
Dual molecular barcode adaptors compared
with single molecular barcode adaptors
provide higher molecular barcode diversity.

| | Expected ratio of |
|---|---|
| Number of barcodes | cfDNA fragment vs. |
| per sequencing read | unique barcode adaptor |
| Single barcode | 1:0.0001 |
| Dual barcodes | 1:1713 |

### Example 5—Analytical Validation of the CRC23 Assay

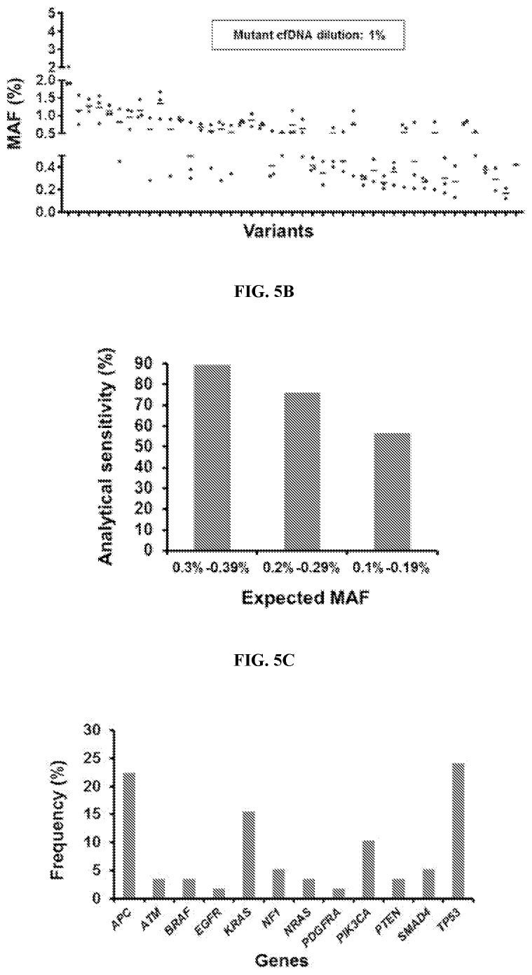

The cfDNA libraries prepared by diluting the HT-29, DLD-1, and MOLT-4 cell line cfDNA pool (mutant) into OCI-AML3 cell line cfDNA (control) at various proportions were sequenced (Table 6). The expected variant allele frequencies in the mutant pool were determined by independently sequencing the cfDNAs used for creating this pool. The sequencing coverage of these variant alleles were from 1116 to 5342 (FIG. 5A). The variant alleles found at 10%, 2%, 1.5%, 1%, 0.5%, and 0.2% dilutions of the mutant pool were compared with the expected variant alleles, and these findings were tabulated into a two-by-two contingency format (Table 7). Analytical accuracy and specificity of the assay were near 100% at all tested mutant pool dilutions. However, assay sensitivity at the 1% dilution was observed to be 86.67% (Table 8). At this dilution, the variant alleles were supposedly distributed between 0.5% and 1%. However, the expected variant frequency calculation indicated that these variants were scattered between 0.16% and 1% (Table 8). In agreement with the expected distribution of variants, the observed frequencies of variants were also distributed widely at all tested dilutions of the mutant pool (FIGS. 5B & 10A-E). Therefore, in order to establish the limit of detection of the assay (defined here as a dilution at which 80% of variants could be detected), the variants that were expected to occur at 0.3-0.39%, 0.2-0.29%, and 0.1-0.19% frequencies were evaluated to determine whether those expected variants could be detected with this assay. The observations indicated that greater than 80% of variants could be identified when they were expected to be present between 0.3 and 0.39% frequencies (FIG. 5C), suggesting that the 0.3% variant frequency was the lower limit of detection of this assay.

TABLE 6
Assembling of a mutant cfDNA pool for library generation. The cfDNAs from HT-
29, DLD-1, and MOLT-4 (mutant) were mixed at equal proportions, and this pool was
diluted at 10%, 2%, 1.5%, 1%, 0.5%, and 0.2% frequencies into the cfDNA from OCI-AML3
(control). Note that 30 ng of cfDNA from each dilution in triplicates was used for library
generation and sequencing.

| cfDNA | | Proportion of Mutant and Control cfDNA | | | | | |
|---|---|---|---|---|---|---|---|
| HT-29 | Mutant | 10% | 2% | 1.5% | 1% | 0.5% | 0.2% |
| DLD-1 | | | | | | | |
| MOLT-4 | | | | | | | |
| | | + | + | + | + | + | + |
| OCI- | Control | 90% | 98% | 98.5% | 99% | 99.5% | 99.8% |
| AML3 | | | | | | | |

TABLE 7
Analytical validation findings from cfDNA mutant
pool diluted at 1%, 0.5%, and 0.2% frequencies.
Note that for orthogonal verification, variants
obtained by independently sequencing each
cfDNA of the mutant pool were used.

| | CRC23 | CRC23 Panel: | | |
|---|---|---|---|---|
| | Panel: | cfDNA from | | |
| | mutant | individual cell lines | | |
| | cfDNA | Positive | Negative | Total |
| 1% | Positive | 117 | 0 | 117 |
| dilution | Negative | 18 | 175935 | 185953 |
| | Total | 135 | 175935 | 176070 |
| 0.5% | Positive | 86 | 0 | 86 |
| dilution | Negative | 49 | 175935 | 175984 |
| | Total | 135 | 175935 | 176070 |
| 0.2% | Positive | 52 | 0 | 52 |
| dilution | Negative | 83 | 175935 | 176018 |
| | Total | 135 | 175935 | 176070 |

TABLE 8
Analytical performance of CRC23 assay at various
mutant allele frequency dilutions. Note that the
expected mutant allele frequencies tend to distribute in a
broader range at each dilution of mutant cfDNA pool.

| Mutant | | | | |
|---|---|---|---|---|
| cfDNA | MAE | Accuracy | Sensitivity | Specificity |
| dilution | (Expected) | (95% CI) | (95% CI) | (95% CI) |
| 10% | 1.56%- | 100% | 100% | 100% |
| | 9.95% | (100%- | (97.30%- | (100%- |
| | | 100%) | 100%) | 100%) |
| 2% | 0.31%- | 100% | 97.04% | 100% |
| | 1.99% | (99.99%- | (92.59%- | (100%- |
| | | 100%) | 99.19%) | 100%) |
| 1.50% | 0.23%- | 100% | 97.78% | 100% |
| | 1.49% | (99.98%- | (93.64%- | (100%- |
| | | 100%) | 99.54%) | 100%) |
| 1% | 0.16%- | 99.99% | 86.67% | 100% |
| | 1.0% | (99.98%- | (79.75%- | (100%- |
| | | 99.99%) | 91.90%) | 100%) |
| 0.50% | 0.08%- | 99.97% | 63.70% | 100% |
| | 0.5% | (99.96%- | (54.99%- | (100%- |
| | | 99.98%) | 71.80%) | 100%) |
| 0.20% | 0.03%- | 99.95% | 38.52% | 100% |
| | 0.2% | (99.94%- | (30.28%- | (100%- |
| | | 99.96%) | 47.28%) | 100%) |

### Example 6—Clinical Validation of the CRC23 Assay

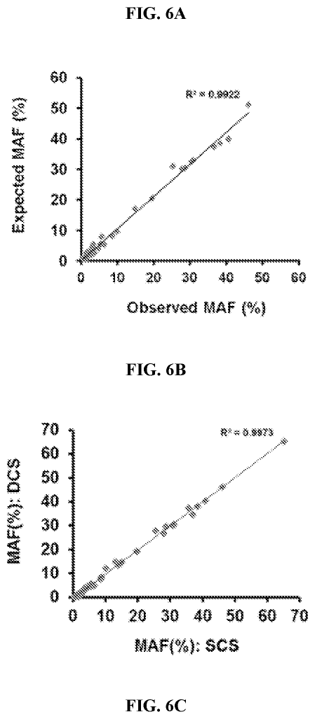

Clinical validation of this assay was performed by sequencing cfDNA samples from 27 patients with colorectal cancer and comparing the findings with the Guardant360 assay findings for orthogonal validation. For comparison purposes, sequencing information from 22 genes that were common to both assays were used, as well as variant alleles at frequencies of 0.3% and above in the Guardant360 assay. APC, KRAS, TP53 were more frequently mutated in the cohort used in this study (FIG. 6A). The diagnostic performance of this assay compared with the Guardant360 assay is shown in two-by-two contingency table format (Table 9), and the findings indicate that the diagnostic accuracy, sensitivity, and specificity of the assay were 96.15%, 87.23%, and 96.91%, respectively (Table 10). The frequencies of variants that were identified in both assays were highly concordant, with an r2 value of 0.99 (FIG. 6B). Variants that were identified exclusively with either the Guardant360 or CRC23 assays were also noted. Interestingly, the variants identified exclusively with the Guardant360 assay had a mutant allele frequency between 0.3 and 0.5, suggesting the variants distributed within this narrow range are only missed in CRC23 assay. Concordance of variant allele frequencies identified from SCS and DCS reads were determined, and the frequencies were highly concordant (r2=0.99) (FIG. 6C).

TABLE 9
Clinical diagnostic performance of CRC23 assay
compared with the Guardant360 assay.

| CRC23 Panel: cfDNA | GH360 Panel: cfDNA NGS | | |
|---|---|---|---|
| duplex sequencing | Positive | Not detected | Total |
| Positive | 41 | 17 | 58 |
| Not detected | 6 | 534 | 540 |
| Total | 47 | 551 | 598 |

TABLE 10
Clinical diagnostic performance of CRC23 assay
compared with the Guardant360 assay.

| | Accuracy | Sensitivity | Specificity |
|---|---|---|---|
| | (95% CI) | (95% CI) | (95% CI) |
| CRC23 vs | 96.15% | 87.23% | 96.91% |
| GH360 | (94.28%- | (74.26%- | (95.11%- |
| | 97.55%) | 95.17%) | 98.19%) |

### Example 7—Longitudinal Monitoring of Disease Progression in Patients with Stage IV Colorectal Cancer Using the CRC23 Assay

To demonstrate a clinical application of this assay, longitudinal monitoring of variant allele frequencies was performed in three plasma samples that were collected from each of five patients at different time points over the treatment course. Variant allele frequency trends were assessed against the inferences of CT scan images obtained during therapy.

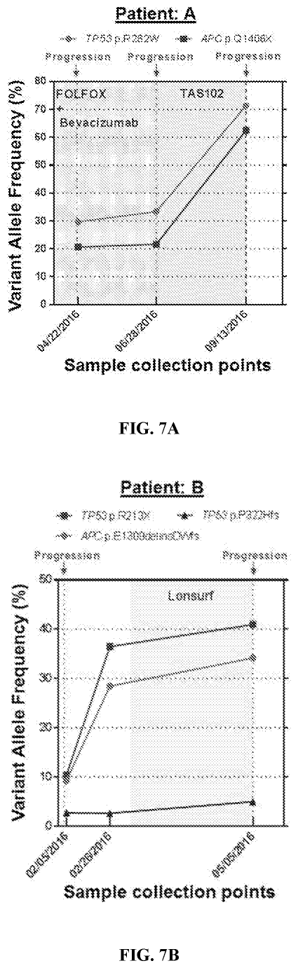

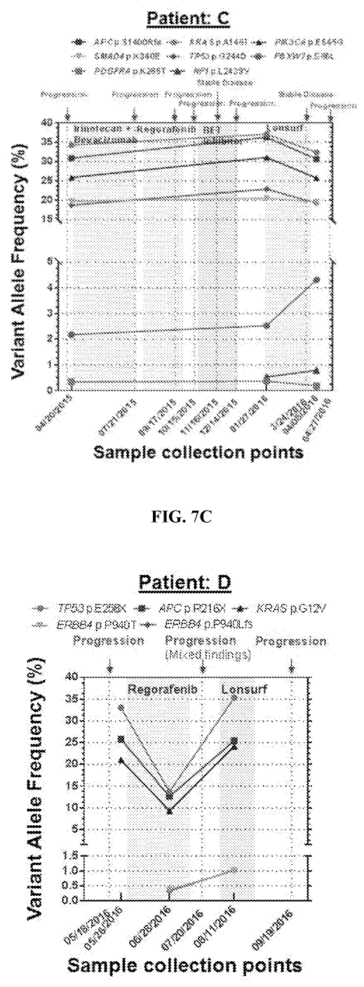

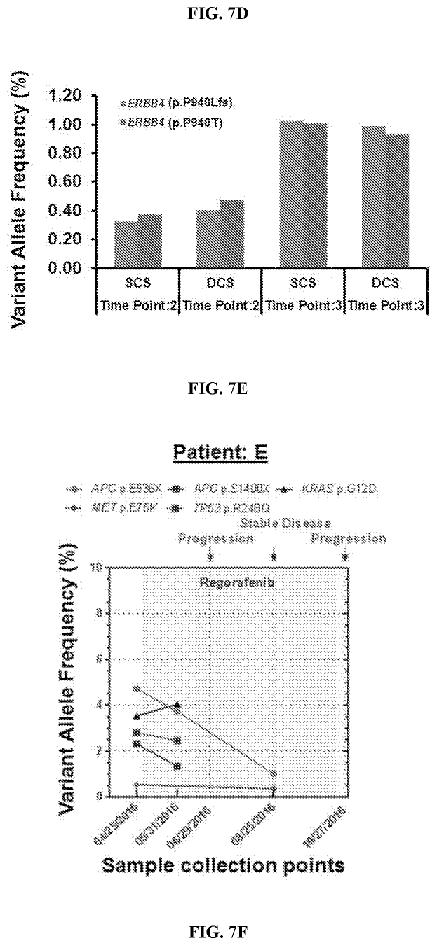

Patient ‘A’ had a primary tumor in the colon and metastases in the liver, adrenal gland, and bone. In the first collected plasma sample, mutant alleles in APC (p.Q1406X) and TP53 (p.R282W) were detected with a frequency greater than 20% (FIG. 7A). The second plasma sample also contained mutant allele frequencies similar to those in the first plasma collection. However, the third collection indicated a 2-fold increase in these two variant allele frequencies. Imaging performed at this collection point also indicated significant progression of the liver, adrenal gland, and bone metastases, suggesting that the observations from cfDNA analysis correlated well with the imaging findings.

In Patient ‘B,’ the primary tumor was located in the colon, with metastases to the liver and lymph nodes. The cfDNA sequencing analysis of the first plasma sample indicated the presence of mutations in APC (p.E1309delinsDW), TP53 (p.R213X), and TP53 (p.P322H) (FIG. 7B). In the second plasma sample, which was collected two weeks after the first collection, the APC (p.E1309delinsDW) and TP53 (p.R213X) mutant allele frequencies were elevated 2-fold. In the third collection, these mutant allele frequencies were observed to be similar to those in the second plasma collection. Imaging performed at this point also indicated mixed treatment responses at diverse metastatic sites, with an overall impression of disease progression.

Patient ‘C’ had a primary tumor in the colon and metastases in the lungs, liver, and lymph nodes. In the first plasma sample, mutations in APC (p.S1400R), KRAS (p.A146T), PIK3CA (p.E545G), SMAD4 (p.K340E), TP53 (p.G244D), FBXW7 (p.S86L), and PDFGRA (p.K265T) were found (FIG. 7C). These mutations and their frequencies remained persistent in the subsequent two plasma collections. Imaging was performed at multiple time points while the patient underwent different treatment regimens. In agreement with the mutant allele frequency observations, imaging performed at different time intervals also indicated advancing disease.

Patient ‘D’ had a primary tumor in the sigmoid colon, with metastases in the liver, peritoneum, and ovary. The first plasma sample contained mutations in the TP53 (p.E258X), APC (p.R216X), and KRAS (p.G12V) and the frequencies of most of these mutant alleles was decreased in the second collection (FIG. 7D). The second sample contained two new mutations in ERBB4, and the accuracy of these variants was further verified through variant calls obtained from DCS reads. Imaging performed close to the time of the second sample collection indicated regression of a few lung lesions and progression in a few other lung sites and the liver. Subsequently, the third plasma sample indicated increased allelic frequencies of most mutants. The imaging performed after the third collection also indicated increases in the size of the lung nodules and liver and peritoneal metastases, which also suggested disease progression.

Patient ‘E’ had a primary tumor in the rectum, with metastases localized in the lungs, liver, lymph nodes, and brain. The first plasma sample was collected prior to initiation of treatment with regorafenib, and the cfDNA analysis indicated the presence of mutations in APC (p.E536X and p.S1400X), KRAS (p.G12D), MET (p.E75K), and TP53 (p.R248Q) genes (FIG. 7E). The second sample was collected 1 month after treatment initiation, and the observed mutant allelic frequencies in this sample were similar to those in the first plasma collection. Imaging performed after the time of second collection also indicated disease progression. The third plasma sample was collected 2 months after the initiation of treatment; mutant allele frequencies in APC (p.E536X and p.S1400X) were reduced and mutations in KRAS (p.G12D) and TP53 (p.R248Q) were not detected. In agreement with these findings, imaging performed on the same day as the third sample collection also indicated stable disease in this patient.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abbosh et al., Phylogenetic ctDNA analysis depicts early-stage lung cancer evolution. Nature 2017, 545:446-451.
Anker & Stroun, Tumor-related alterations in circulating DNA, potential for diagnosis, prognosis and detection of minimal residual disease. Leukemia 2001, 15:289-291.
Arbeithuber et al., Artifactual mutations resulting from DNA lesions limit detection levels in ultrasensitive sequencing applications. DNA Res 2016, 23:547-559.
Bettegowda et al., Detection of circulating tumor DNA in early- and late-stage human malignancies. Sci Transl Med 2014, 6:224ra224.
Bruskov et al., Heat-induced formation of reactive oxygen species and 8-oxoguanine, a biomarker of damage to DNA. Nucleic Acids Res 2002, 30:1354-1363.
Cassinotti et al., Free circulating DNA as a biomarker of colorectal cancer. Int J Surg 2013, 11 Suppl 1:S54-57.
Castro-Giner et al., Cancer Diagnosis Using a Liquid Biopsy: Challenges and Expectations. Diagnostics (Basel) 2018, 8.
Christensen et al., Optimized targeted sequencing of cell-free plasma DNA from bladder cancer patients. Sci Rep 2018, 8:1917.
Diehl et al., Circulating mutant DNA to assess tumor dynamics. Nat Med 2008, 14:985-990.
Foubert et al., Options for metastatic colorectal cancer beyond the second line of treatment. Dig Liver Dis 2014, 46:105-112.
Frenel et al., Serial Next-Generation Sequencing of Circulating Cell-Free DNA Evaluating Tumor Clone Response To Molecularly Targeted Drug Administration. Clin Cancer Res 2015, 21:4586-4596.
Garcia-Garcia et al., Assessment of the latest NGS enrichment capture methods in clinical context. Sci Rep 2016, 6:20948.
Guo et al., Circulating tumor DNA detection in lung cancer patients before and after surgery. Sci Rep 2016, 6:33519.
Hao et al., Circulating cell-free DNA in serum as a biomarker for diagnosis and prognostic prediction of colorectal cancer. Br J Cancer 2014, 111:1482-1489.
Heitzer et al., The potential of liquid biopsies for the early detection of cancer. NPJ Precis Oncol 2017, 1:36.
Hench et al., Liquid Biopsy in Clinical Management of Breast, Lung, and Colorectal Cancer. Front Med (Lausanne) 2018, 5:9.
Kamps-Hughes et al., ERASE-Seq: Leveraging replicate measurements to enhance ultralow frequency variant detection in NGS data. PLoS One 2018, 13:e0195272.
Kennedy et al., Detecting ultralow-frequency mutations by Duplex Sequencing. Nat Protoc 2014, 9:2586-2606.
Kidess et al., Mutation profiling of tumor DNA from plasma and tumor tissue of colorectal cancer patients with a novel, high-sensitivity multiplexed mutation detection platform. Oncotarget 2015, 6:2549-2561.
Lanman et al., Analytical and Clinical Validation of a Digital Sequencing Panel for Quantitative, Highly Accurate Evaluation of Cell-Free Circulating Tumor DNA. PLoS One 2015, 10:e0140712.
Lebofsky et al., Circulating tumor DNA as a non-invasive substitute to metastasis biopsy for tumor genotyping and personalized medicine in a prospective trial across all tumor types. Mol Oncol 2015, 9:783-790.
Ma et al., “Liquid biopsy”-ctDNA detection with great potential and challenges. Ann Transl Med 2015, 3:235.
Mehrotra et al., Study of Preanalytic and Analytic Variables for Clinical Next-Generation Sequencing of Circulating Cell-Free Nucleic Acid. J Mol Diagn 2017, 19:514-524.
Newman et al., Integrated digital error suppression for improved detection of circulating tumor DNA. Nat Biotechnol 2016, 34:547-555.
Norton et al., A stabilizing reagent prevents cell-free DNA contamination by cellular DNA in plasma during blood sample storage and shipping as determined by digital PCR. Clin Biochem 2013, 46:1561-1565.
Park et al., Characterization of background noise in capture-based targeted sequencing data.

Genome Biol 2017, 18:136.
Pereira et al., Clinical utility of circulating cell-free DNA in advanced colorectal cancer. PLoS One 2017, 12:e0183949.
Robasky et al., The role of replicates for error mitigation in next-generation sequencing. Nat Rev Genet 2014, 15:56-62.
Salk et al., Enhancing the accuracy of next-generation sequencing for detecting rare and subclonal mutations. Nat Rev Genet 2018, 19:269-285.
Samorodnitsky et al., Evaluation of Hybridization Capture Versus Amplicon-Based Methods for Whole-Exome Sequencing. Hum Mutat 2015, 36:903-914.
Schmitt et al., Detection of ultra-rare mutations by next-generation sequencing. Proc Natl Acad Sci USA 2012, 109:14508-14513.
Scholer et al., Clinical Implications of Monitoring Circulating Tumor DNA in Patients with Colorectal Cancer. Clin Cancer Res 2017, 23:5437-5445.
Schrock et al., Hybrid Capture-Based Genomic Profiling of Circulating Tumor DNA from Patients with Advanced Cancers of the Gastrointestinal Tract or Anus. Clin Cancer Res 2018, 24:1881-1890.
Shu et al., Circulating Tumor DNA Mutation Profiling by Targeted Next Generation Sequencing Provides Guidance for Personalized Treatments in Multiple Cancer Types. Sci Rep 2017, 7:583.
Stoler et al., Streamlined analysis of duplex sequencing data with Du Novo. Genome Biol 2016, 17:180.
Strickler et al., Genomic Landscape of Cell-Free DNA in Patients with Colorectal Cancer. Cancer Discov 2018, 8:164-173.
Stroun et al., About the possible origin and mechanism of circulating DNA apoptosis and active DNA release. Clin Chim Acta 2001, 313:139-142.
Takai et al., Clinical utility of circulating tumor DNA for molecular assessment in pancreatic cancer. Sci Rep 2015, 5:18425.
Thierry et al., Origins, structures, and functions of circulating DNA in oncology. Cancer Metastasis Rev 2016, 35:347-376.
Thierry et al., Circulating DNA Demonstrates Convergent Evolution and Common Resistance Mechanisms during Treatment of Colorectal Cancer. Clin Cancer Res 2017, 23:4578-4591.
Tie et al., Circulating tumor DNA as an early marker of therapeutic response in patients with metastatic colorectal cancer. Ann Oncol 2015, 26:1715-1722.
Tie et al., Circulating tumor DNA analysis detects minimal residual disease and predicts recurrence in patients with stage II colon cancer. Sci Transl Med 2016, 8:346ra392.
Tie et al., Serial circulating tumour DNA analysis during multimodality treatment of locally advanced rectal cancer: a prospective biomarker study. Gut 2019, 68: 663-671.
To et al., Rapid clearance of plasma Epstein-Barr virus DNA after surgical treatment of nasopharyngeal carcinoma. Clin Cancer Res 2003, 9:3254-3259.
Underhill et al., Fragment Length of Circulating Tumor DNA. PLoS Genet 2016, 12:e1006162.
Wan et al., Liquid biopsies come of age: towards implementation of circulating tumour DNA. Nat Rev Cancer 2017, 17:223-238.
Williams et al., A high frequency of sequence alterations is due to formalin fixation of archival specimens. Am J Pathol 1999, 155:1467-1471.
Yao et al., Evaluation and comparison of in vitro degradation kinetics of DNA in serum, urine and saliva: A qualitative study. Gene 2016, 590:142-148.
Zhou et al., Application of Circulating Tumor DNA as a Non-Invasive Tool for Monitoring the Progression of Colorectal Cancer. PLoS One 2016, 11:e0159708.

What is claimed is:

1. A method of preparing a library of cell-free DNA (cfDNA) for sequencing, the method comprising:
(a) obtaining a sample comprising a plurality of cfDNA;
(b) performing end-repair and A-tailing reactions on between about 5 ng and about 30 ng of the plurality of cfDNA in a reaction having a first reaction volume;
(c) contacting between about 2.5 ng and about 15 ng of the plurality of cfDNA with a population of stem-loop adaptors and a ligase in a second reaction volume that is about equal to the first reaction volume, wherein the stem-loop adaptors each comprise an inverted repeat and a loop, wherein the loop comprises at least one cleavable base, thereby ligating a stem-loop adaptor to each end of the plurality of cfDNA to produce adaptor-ligated cfDNA;
(d) linearizing the adaptor-ligated cfDNA by cleaving the cleavable base;
(e) amplifying the linearized adaptor-ligated cfDNA to produce amplified adaptor-ligated cfDNA, wherein the amplification uses forward and reverse primers complementary to known sequences in the stem-loop adaptors;
(f) contacting the amplified adaptor-ligated cfDNA with RNA baits that hybridize to selected molecules of the plurality of cfDNA, wherein the weight ratio of RNA baits:amplified adaptor-ligated cfDNA is between about 1:25 and about 1:250;
(g) isolating the molecules of the plurality of cfDNA having a hybridized RNA bait, thereby producing enriched cfDNA; and
(h) amplifying the enriched cfDNA with indexing primers, thereby producing a library of cfDNA for sequencing.

2. The method of claim 1, wherein the method maintains variant allele frequencies in the cfDNA.

3. The method of any one of claims 1-2, wherein the cfDNA comprises double-stranded DNA molecules.

4. The method of any one of claims 1-3, wherein the cfDNA is obtained from a body fluid.

5. The method of claim 4, wherein the body fluid comprises blood, serum, urine, cerebrospinal fluid, nipple aspirate, sweat, or saliva.

6. The method of any one of claims 1-5, wherein the cfDNA is obtained from an individual having a cancer.

7. The method of any one of claims 1-6, wherein end repair comprises exposing the plurality of cfDNA to a terminal deoxynucleotidyltransferase and an adenine deoxyribonucleotide.

8. The method of any one of claims 1-7, wherein the stem-loop adaptors comprise a 3′ T overhang.

9. The method of any one of claims 1-8, wherein the stem-loop adaptors comprise a 3′ hydroxyl and a 5′ phosphate.

10. The method of any one of claims 1-9, wherein the population of stem-loop adaptors comprises 75 ng of stem-loop adaptors.

11. The method of any one of claims 1-10, wherein the stem-loop adaptors each comprise a constant region having a known sequence that is constant among the population of stem-loop adaptors and a barcode region having a sequence that is degenerate among the population of stem-loop adaptors.

12. The method of claim 11, wherein the barcode region is 4 nucleotides to 20 nucleotides in length.

13. The method of claim 12, wherein the barcode region is 14 nucleotides in length.

14. The method of any one of claims 11-13, wherein the barcode region is in the inverted repeat.

15. The method of any one of claims 11-14, wherein the barcode regions are sufficiently unique so that each tagged double-stranded cfDNA molecule can be differentiated from other tagged double-stranded cfDNA molecules.

16. The method of any one of claims 11-15, wherein the barcode regions of the stem-loop adaptors attached to each end of a cfDNA molecule comprise unique sequences.

17. The method of any one of claims 1-16, wherein the cleavable base is deoxyuridine.

18. The method of any one of claims 1-17, wherein the cleavable base is cleaved prior to step (e).

19. The method of any one of claims 1-18, wherein step (f) further comprises contacting the amplified adaptor-ligated cfDNA with adaptor blockers.

20. The method of any one of claims 1-19, wherein the RNA baits hybridize to selected genomic loci in a reference genome.

21. The method of claim 20, wherein the hybridization of the RNA baits to the cfDNA selectively enriches the cfDNA for strands that map to said genomic loci.

22. The method of any one of claims 20-21, wherein the selected genomic loci comprise disease-associated genetic loci.

23. The method of any one of claims 20-22, wherein the selected genomic loci comprise cancer-associated genetic loci.

24. The method of any one of claims 20-23, wherein the selected genomic loci are in genes selected from the group consisting of TP53, APC, ATM, KRAS, NRAS, BRAF, PIK3CA, EGFR, NF1, NRAS, PDGFRA, PTEN, SMAD4, and ERBB2.

25. The method of any one of claims 1-24, wherein the RNA baits are oligonucleotides between about 70 nucleotides and 1000 nucleotides in length.

26. The method of any one of claims 1-25, wherein the target-specific sequences in the RNA baits are between about 100 and about 200 nucleotides in length.

27. The method of any one of claims 1-26, wherein the RNA baits have sequences that hybridize to a target sequence for at least 50 of the genomic loci listed in Table 1.

28. The method of any one of claims 1-27, wherein the RNA baits each comprise an affinity tag.

29. The method of claim 28, wherein the affinity tag is a biotin molecule or a hapten.

30. The method of any one of claims 1-29, wherein step (g) comprises contacting the hybridized molecules from step (f) with a molecule or particle that binds to the RNA baits and isolating the RNA bait sequences, thereby isolating the subgroup of cfDNA molecules that hybridized to the RNA baits.

31. The method of claim 30, wherein the molecule or particle that binds to the RNA baits binds to the affinity tag.

32. The method of claim 30, wherein the molecule or particle that binds to the RNA baits is an avidin molecule or an antibody that binds to the hapten.

33. The method of any one of claims 1-32, wherein amplifying in step (e) and/or (h) comprises performing polymerase chain reaction.

34. A library of cfDNA molecules generated by the method of any one of claims 1-33.

35. A method of analyzing the library of cfDNA molecules of claim 34, comprising (a) sequencing the library of cfDNA.

36. The method of claim 35, further comprising (b) generating a single consensus sequence for each forward and reverse sequence by grouping all sequencing reads that share the same variant adaptor sequences on both their 5′ and 3′ ends, representing each position in the consensus sequence with the nucleotide present in the sequencing reads only if all sequencing reads in the family have the same nucleotide at that position, representing each position in the consensus sequence with N if the sequencing reads in the family have different nucleotides at that position.

37. The method of claim 36, further comprising generating a double consensus sequence by (a) identifying a reverse single consensus sequence having a molecular barcode in reverse orientation relative to a molecular barcode for a given forward single consensus sequence, representing each position in the double consensus sequence with the nucleotide present in both the forward SCS and reverse SCS reads only if the forward SCS and reverse SCS have the same nucleotide at that position, representing each position in the DCS with N if the forward SCS and the reverse SCS have different nucleotides at that position; and (b) identifying a forward single consensus sequence having a molecular barcode in reverse orientation relative to a molecular barcode for a given reverse single consensus sequence, representing each position in the double consensus sequence with the nucleotide present in both the forward SCS and reverse SCS reads only if the forward SCS and reverse SCS have the same nucleotide at that position, representing each position in the DCS with N if the forward SCS and the reverse SCS have different nucleotides at that position.

38. The method of claim 36, further comprising aligning the single consensus sequences derived from families containing at least two reads with a human reference genome and identifying variants in the single consensus sequences.

39. The method of claim 37, further comprising aligning the double consensus sequences with a human reference genome and identifying variants in the double consensus sequences.

40. The method of any one of claims 35-39, further comprising detecting a copy number variation in the cfDNA, wherein the copy number variation is based at least on part on the quantification of the sequencing reads that map to each of one or more genetic loci.

41. The method of any one of claims 35-40, further comprising quantifying cfDNA molecules bearing a sequence variant.

42. The method of claim 41, wherein quantifying cfDNA molecules bearing a sequence variant comprises only counting the variant allele if the variant allele count was at least 4.

43. The method of claim 41, wherein quantifying cfDNA molecules bearing a sequence variant comprises only counting the variant allele if the read balance ratio was at least 0.1.

44. The method of claim 41, wherein quantifying cfDNA molecules bearing a sequence variant comprises only counting the variant allele if the ratio of variant frequency in the sample is more than two-fold different than a variant frequency in a healthy control sample.

45. A method of monitoring progression of cancer in a patient, monitoring response to therapy in a cancer patient, or detecting minimum residual disease in a cancer patient, the method comprising analyzing cfDNA obtaining from the patient at at least two time points according to the method of any one of claims 35-44 and comparing the variant allele frequencies at the at least two time points.

46. The method of claim 45, wherein the patient has colorectal cancer, ovarian cancer, lung cancer, prostate cancer, liver cancer, kidney cancer, pancreatic cancer, uterine cancer, brain cancer, skin cancer, stomach cancer, or breast cancer.

47. A composition comprising a set of RNA baits that hybridize to a target sequence for at least 50 of the genomic loci listed in Table 1.

48. The composition of claim 47, wherein the composition comprises RNA baits that hybridize to the target sequence for at least 100, 150, 200, or 250 of the genomic loci listed in Table 1.

49. The composition of claim 47, wherein the composition comprises RNA baits that hybridize to the target sequence of all 274 of the genomic loci listed in Table 1.

50. The composition of any one of claims 47-49, wherein the RNA baits each comprise an affinity tag.


SEQUENCE LISTING (4 sequences)

SEQ ID NO: 1 - DNA, 13 bp, Artificial sequence
  Synthetic oligonucleotide
  misc_feature (1)..(3): n is a, c, g, or t
  misc_feature (5)..(8): n is a, c, g, or t
  misc_feature (10)..(13): n is a, c, g, or t
nnncnnnntnnnn

SEQ ID NO: 2 - DNA, 14 bp, Artificial sequence
  Synthetic oligonucleotide
  misc_feature (1)..(4): n is a, c, g, or t
  misc_feature (6)..(9): n is a, c, g, or t
  misc_feature (11)..(14): n is a, c, g, or t
nnnncnnnntnnnn

SEQ ID NO: 3 - DNA, 14 bp, Artificial sequence
  Synthetic oligonucleotide
  misc_feature (1)..(4): n is a, c, g, or t
  misc_feature (6)..(9): n is a, c, g, or t
  misc_feature (11)..(14): n is a, c, g, or t
nnnnannnngnnnn

SEQ ID NO: 4 - DNA, 20 bp, Artificial sequence
  Synthetic oligonucleotide
cacttttcaacatagtgtgg